(19) United States
(12) Patent Application Publication
(10) Pub. No.: US 2017/0156884 A1
(43) Pub. Date: Jun. 8, 2017

(54) PLATE CAGE SYSTEM WITH STANDALONE EFFECTS AND RELATED METHODS

(71) Applicant: CTL Medical Corporation, Addison, TX (US)

(72) Inventors: David S. Rathbun, Gap, PA (US); Sean Suh, East Brunswick, NJ (US)

(21) Appl. No.: 15/173,130

(22) Filed: Jun. 3, 2016

Related U.S. Application Data
(60) Provisional application No. 62/264,183, filed on Dec. 7, 2015.
(60) Provisional application No. 62/264,496, filed on Dec. 8, 2015.

(51) Int. Cl. A61F 2/44

(52) U.S. Cl. CPC A61F 2/4425; A61F 2/442

(57) ABSTRACT

A cage for implanting in bone that has a first plate having a surface that contacts a first bone surface, a second plate having a surface that contacts a second bone surface, a intermediary plate that dynamically couples to the first plate and the second plate, an actuator that drives and causes the intermediary plate to move between the first plate and the second plate along a predetermined direction, and an anchor that attaches to the first plate and the second plate to engage the actuator to drive the actuator longitudinally along the predetermined direction.

### CROSS-REFERENCE TO RELATED APPLICATIONS

This application claims priority to and benefit thereof from U.S. Provisional Patent Application No. 62/264,183, filed Dec. 7, 2015, and U.S. Provisional Patent Application No. 62/264,496, filed Dec. 8, 2015, both titled “PLATE AND CAGE SYSTEM WITH STANDALONE EFFECTS AND RELATED METHODS,” and both of which are hereby incorporated herein by reference in their entireties.

### FIELD OF THE DISCLOSURE

The present disclosure relates to intervertebral and intradiscal implants and related systems and methods. More specifically, the present disclosure relates to intervertebral and intradiscal devices, systems, and methods for deployment within a body of a patient.

### BACKGROUND OF THE DISCLOSURE

In mammals, the spinal (or vertebral) column is one of the most important parts. The spinal column provides the main support necessary for mammals to stand, bend, and twist.

In humans, the spinal column is generally formed by individual interlocking vertebrae, which are classified into five segments, including (from head to tail) a cervical segment (vertebrae C1-C7), a thoracic segment (vertebrae T1-T12), a lumbar segment (vertebrae L1-L5), a sacrum segment (vertebrae S1-S5), and coccyx segment (vertebrate Co1-Co5). The cervical segment forms the neck, supports the head and neck, and allows for nodding, shaking and other movements of the head. The thoracic segment attaches to ribs to form the ribcage. The lumbar segment carries most of the weight of the upper body and provides a stable center of gravity during movement. The sacrum and coccyx make up the back walls of the pelvis.

Intervertebral discs are located between each of the movable vertebra. Each intervertebral disc typically includes a thick outer layer called the disc annulus, which includes a crisscrossing fibrous structure, and a disc nucleus, which is a soft gel-like structure located at the center of the disc. The intervertebral discs function to absorb force and allow for pivotal movement of adjacent vertebra with respect to each other.

In the vertebral column, the vertebrae increase in size as they progress from the cervical segment to the sacrum segment, becoming smaller in the coccyx. At maturity, the five sacral vertebrae typically fuse into one large bone, the sacrum, with no intervertebral discs. The last three to five coccygeal vertebrae (typically four) form the coccyx (or tailbone). Like the sacrum, the coccyx does not have any intervertebral discs.

Each vertebra is an irregular bone that varies in size according to its placement in the spinal column, spinal loading, posture and pathology. While the basic configuration of vertebrae varies, every vertebra has a body that consists of a large anterior middle portion called the centrum and a posterior vertebral arch called the neural arch. The upper and lower surfaces of the vertebra body give attachment to intervertebral discs. The posterior part of a vertebra forms a vertebral arch that typically consists of two pedicles, two laminae, and seven processes. The laminae give attachment to the ligament flava, and the pedicles have a shape that forms vertebral notches to form the intervertebral foramina when the vertebrae articulate. The foramina are the entry and exit passageways for spinal nerves. The body of the vertebra and the vertical arch form the vertebral foramen, which is a large, central opening that accommodates the spinal canal that encloses and protects the spinal cord.

The body of each vertebra is composed of cancellous bone that is covered by a thin coating of cortical bone. The cancellous bone is a spongy type of osseous tissue, and the cortical bone is a hard and dense type of osseous tissue. The vertebral arch and processes have thicker coverings of cortical bone.

The upper and lower surfaces of the vertebra body are flattened and rough. These surfaces are the vertebral endplates that are in direct contact with the intervertebral discs. The endplates are formed from a thickened layer of cancellous bone, with the top layer being denser. The endplates contain adjacent discs and evenly spread applied loads. The endplates also provide anchorage for the collagen fibers of the disc.

FIG. 1 shows a portion of a patient's spinal column 2, including vertebra 4 and intervertebral discs 6. As noted earlier, each disc 6 forms a fibrocartilaginous joint between adjacent vertebrae 4 so as to allow relative movement between adjacent vertebrae 4. Beyond enabling relative motion between adjacent vertebrae 4, each disc 6 acts as a shock absorber for the spinal column 2.

As noted earlier, each disc 6 comprises a fibrous exterior surrounding an inner gel-like center which cooperate to distribute pressure evenly across each disc 6, thereby preventing the development of stress concentrations that might otherwise damage and/or impair vertebrae 4 of spinal column 2. Discs 6 are, however, subject to various injuries and/or disorders which may interfere with a disc's ability to adequately distribute pressure and protect vertebrae 4. For example, disc herniation, degeneration, and infection of discs 6 may result in insufficient disc thickness and/or support to absorb and/or distribute forces imparted to spinal column 2. Disc degeneration, for example, may result when the inner gel-like center begins to dehydrate, which may result in a degenerated disc 8 having decreased thickness. This decreased thickness may limit the ability of degenerated disc 8 to absorb shock which, if left untreated, may result in pain and/or vertebral injury.

While pain medication, physical therapy, and other non-operative conditions may alleviate some symptoms, such interventions may not be sufficient for every patient. Accordingly, various procedures have been developed to surgically improve patient quality of life via abatement of pain and/or discomfort. Such procedures may include, discectomy and fusion procedures, such as, for example, anterior cervical interbody fusion (ACIF), anterior lumbar interbody fusion (ALIF), direct lateral interbody fusion (DLIF) (also known as XLIF), posterior lumbar interbody fusion (PLIF), and transforaminal lumbar interbody fusion (TLIF). During a discectomy, all or a portion of a damaged disc (for example, degenerated disc 8, shown in FIG. 1), is removed via an incision, typically under X-ray guidance.

Following the discectomy procedure, a medical professional may determine an appropriate size of an interbody device 9 (shown in FIG. 2) via one or more distractors and/or trials of various sizes. Each trial and/or distractor may be forcibly inserted between adjacent vertebrae 4. Upon determination of an appropriate size, one or more of an ACIF, ALIF, DLIF, PLIF, and/or TLIF may be performed by placing an appropriate interbody device 9 (such as, for example, a cage, a spacer, a block) between adjacent vertebrae 4 in the space formed by the removed degenerated disc 8. Placement of such interbody devices 9 within spinal column 2 may prevent spaces between adjacent vertebrae 4 from collapsing, thereby preventing adjacent vertebrae 4 from resting immediately on top of one another and inducing fracture of vertebra 4, impingement of the spinal cord, and/or pain. Additionally, such interbody devices 9 may facilitate fusion between adjacent vertebrae 4 by stabilizing adjacent vertebrae 4 relative to one another. Accordingly, as shown in FIG. 2, such interbody devices 9 often may include one or more bone screws 11 extending through interbody device 9 and into adjacent vertebrae 4.

Often, following the removal of the distractor and/or trial, a medical professional must prepare one or more bores or holes in a vertebra 4 intended to receive the bone screws 11. Such holes may be formed with the aid of a separate drill guide positioned proximate or abutting vertebra 4 and inserting a drill therethrough. Alternatively, such holes may be formed free hand, without the use of a drill guide. Further, since spinal column 2 is subject to dynamic forces, often changing with each slight movement of the patient, such screw(s) 11 have a tendency to back out (for example, unscrew) and/or dislodge from interbody device 9, thereby limiting interbody device's 9 ability to stabilize adjacent vertebrae 4, and consequently, promote fusion. Additionally, if screw(s) 11 back out and/or dislodge from the interbody device 9, they may inadvertently contact, damage, and/or irritate surrounding tissue. Further, interbody device 9 is commonly comprised of a radiopaque material so as to be visible in situ via x-ray and other similar imaging modalities. However, such materials may impede sagittal and/or coronal visibility, thereby preventing visual confirmation of placement and post-operative fusion.

Thus, there remains a need for improved interbody devices, associated systems, and methodologies related thereto.

### SUMMARY OF THE DISCLOSURE

The present disclosure includes examples that relate to, among other things, intradiscal, extradiscal, or interdiscal implants. The cages, plating devices, and cage systems disclosed herein may be used as, for example, but not limited to, standalone anterior lumbar interbody fusion devices, standalone anterior low-profile plating devices, an interlocking of standalone devices to create hybrid devices, modular systems to allow interchangeability, and the like. Each of the examples disclosed herein may include one or more features described in connection with any of the other disclosed examples.

According to a non-limiting aspect of the disclosure, a cage for implanting in bone, comprises: a first plate having a surface that contacts a first bone surface; a second plate having a surface that contacts a second bone surface; an intermediary plate that dynamically couples to the first plate and the second plate; an actuator that drives and causes the intermediary plate to move between the first plate and the second plate along a predetermined direction; and an anchor that attaches to the first plate and the second plate to engage the actuator to drive the actuator longitudinally along the predetermined direction. The cage may further comprise a pin that engages an anterior portion of the intermediary plate. The pin may engage a portion of the actuator to substantially affix the actuator to the intermediary plate. At least one of the first plate and second plate may comprise a guide track that engages and guides the intermediary plate as it moves between the first plate and the second plate along the predetermined direction. The intermediary plate may comprise a guide that engages the guide track to go guide the intermediary plate as it moves between and along inner surfaces of the first plate and the second plate in the predetermined direction. The anchor may comprise an anchor lock that engages the first plate or the second plate to prevent the anchor from moving, which, otherwise, may comprise rotation of the anchor about a longitudinal axis of the actuator. At least one of the first plate and the second plate may comprise a receiver that holds the anchor lock. The inner walls of the first plate, second plate and intermediary plate may form one or more graft chambers.

According to a further aspect of the disclosure, a cage for implanting in bone comprises: a first plate having a surface that contacts a first bone surface; a second plate having a surface that contacts a second bone surface; a intermediary plate that movably attaches to the first plate and the second plate; and an actuator that drives and causes the intermediary plate to move between the first plate and the second plate along a predetermined direction. The cage may further comprise an anchor that engages the actuator to drive the actuator longitudinally along the predetermined direction, or in a direction substantially opposite to the predetermined direction. At least one of the first plate and second plate comprises a guide track that engages and guides the intermediary plate as it moves along the predetermined direction between the first plate and the second plate. The intermediary plate may comprise a guide that engages the guide track to go guide the intermediary plate as it moves between the first plate and the second plate along the predetermined direction. The anchor may comprise an anchor lock that engages at least one of the first plate and the second plate to prevent the anchor from moving, which may comprise rotation of the anchor about a longitudinal axis of the actuator. The inner walls of the first plate, second plate and intermediary plate may form a graft chamber. At least one of the first plate and the second plate may comprise a receiver that holds the anchor lock. The cage may further comprise a pin that engages and holds an anterior portion of the intermediary plate with respect to a portion of the actuator.

According to a still further aspect of the disclosure, a cage for implanting in bone comprises: a first plate having a surface that contacts a bone surface; an intermediary plate that movably attaches to the first plate; and an actuator that drives and causes the intermediary plate to move with respect to the first plate along a predetermined direction. The cage may further comprise a second plate having a surface that contacts another bone surface, wherein the intermediary plate movably attaches to the second plate.

Additional features, advantages, and embodiments of the disclosure may be set forth or apparent from consideration of the detailed description and drawings. Moreover, it is to be understood that both the foregoing summary of the disclosure and the following detailed description are exemplary and intended to provide further explanation without limiting the scope of the disclosure as claimed.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to help explain the principles of the disclosure. No attempt is made to show structural details of the disclosure in more detail than may be necessary for a fundamental understanding of the disclosure and the various ways in which it may be practiced. In the drawings:

FIG. 1 illustrates a portion of a patient's spinal column;

FIG. 2 illustrates an interbody device positioned within the patient's spinal column;

FIGS. 3A-3C illustrate perspective, side, and top (or bottom) views, respectively, of a cage that is constructed according to the principles of the disclosure;

FIG. 4 illustrates a side cross-section view of the cage;

FIGS. 5-6 illustrate perspective and side views, respectively, of the cage with an intermediary plate partially removed from (or installed in) a plate system;

FIG. 7 illustrates a perspective view of the cage with an exemplary plating device coupled to the intermediary plate, showing the intermediary plate partially removed from (or installed in) the plate system;

FIG. 8 illustrates a perspective view of the cage with the exemplary plating device coupled to the intermediary plate, showing the intermediary plate substantially completely installed in the plate system;

FIG. 9 illustrates a perspective side view of the plating device in FIGS. 7-8.

FIG. 10 illustrates another example of a plating device that may be coupled to the cage;

FIG. 11 illustrates the plating device of FIGS. 7-9 coupled to the cage and provided with bone fasteners;

FIG. 12 illustrates the plating device of FIG. 9 coupled to the cage and provided with bone fasteners;

FIG. 13 illustrates an exploded view of the plating device and cage of FIG. 12;

FIG. 14 illustrates a cut-away view of the plating device and cage of FIG. 12, provided with one or more channels;

FIG. 15A illustrates a cut-away view of the plating device and cage of FIG. 12, provided with one or more channels, including at least one channel that flows into a graft chamber;

FIGS. 15B and 15C illustrate an example of a fastener blocking mechanism that may be included in the plating device;

FIGS. 15D and 15E illustrate an example of a drop-down assembly design that may be implemented for the plating device and cage;

FIGS. 15F and 15G illustrate an example of a rotate and lock assembly design that may be implemented for the plating device and cage;

FIGS. 16-18 illustrate various stages of installing the cage with plating device in a patient;

FIGS. 19-21 illustrate perspective, side, and front views of the cage with plating device, including bone fasteners;

FIGS. 22-25 illustrate various stages of installing the cage with plating device in a patient;

FIGS. 26-28 illustrate perspective and top (or bottom) views of another example of a cage that is constructed according to the principles of the disclosure;

FIGS. 29-30 illustrate various stages of installing the cage of FIGS. 26-28 in a patient;

FIGS. 31-32 illustrate perspective and top (or bottom) views, respectively, of another example of a cage;

FIGS. 33-34 illustrate perspective and top (or bottom) views, respectively, of yet another example of a cage that is constructed according to the principles of the disclosure;

FIGS. 35-36 illustrate perspective and top (or bottom) views, respectively, of a further example of a cage that is constructed according to the principles of the disclosure;

FIG. 37 illustrates a side view of the cage of FIGS. 31-36;

FIG. 38 illustrates the cage according to FIGS. 31-36, installed in a patient;

FIGS. 39-41 illustrate perspective, side and top (or bottom) views, respectively, of a still further example of a cage that is constructed according to the principles of the disclosure;

FIGS. 42-44 illustrate perspective, side and top (or bottom) views, respectively, of a still further example of a cage that is constructed according to the principles of the disclosure;

FIGS. 45-47 illustrate various stages of installing the cage of FIGS. 42-44 in a patient;

FIGS. 48-49 illustrate perspective and side views, respectively, of still a further example of a cage that is constructed according to the principles of the disclosure; and

FIGS. 50-51 illustrate various stages of installing the cage of FIGS. 48-49 in a patient.

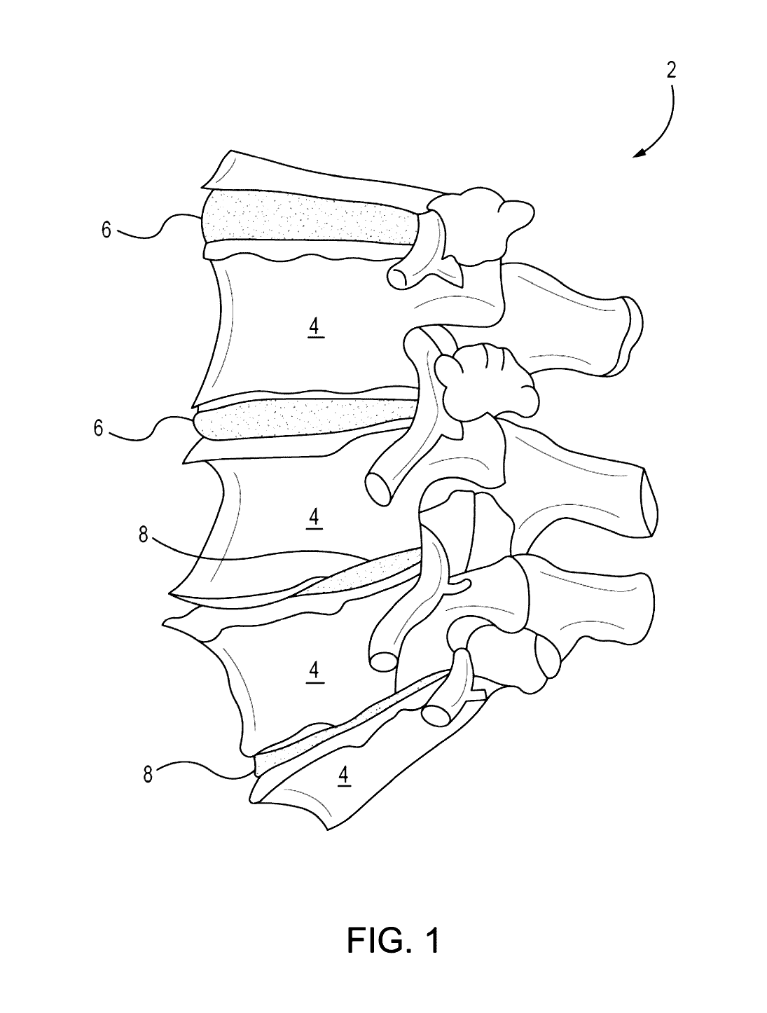

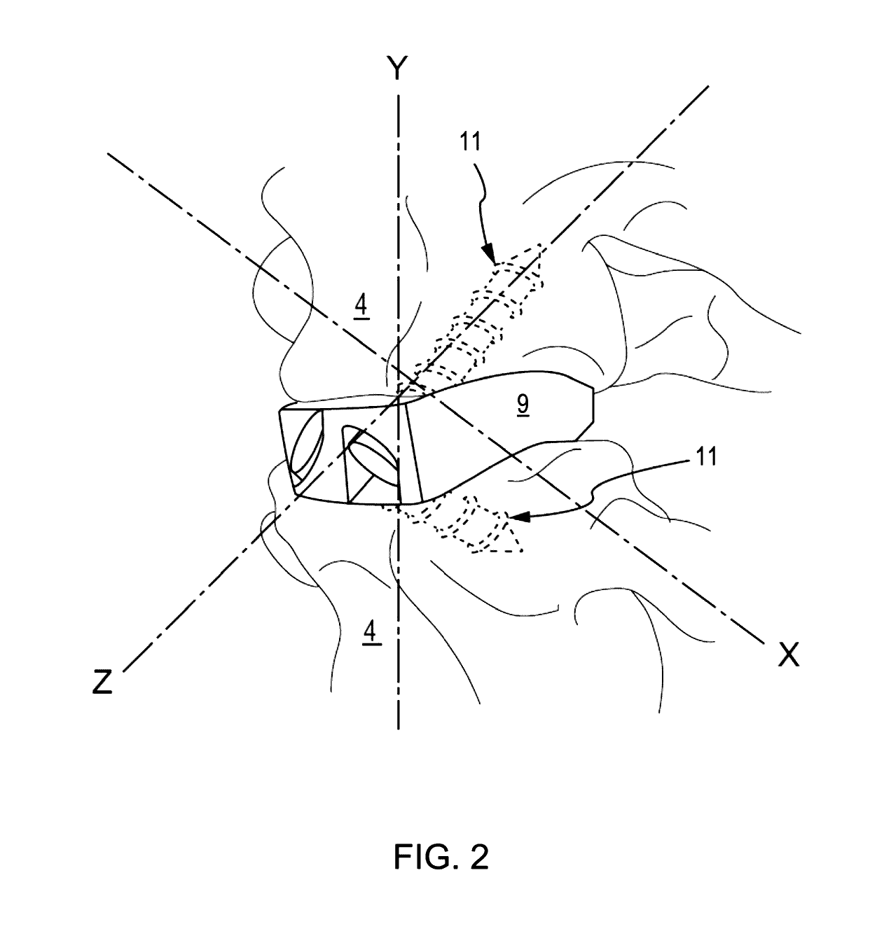

The present disclosure is further described in the detailed description that follows.

### DETAILED DESCRIPTION OF THE DISCLOSURE

The disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals represent similar parts throughout the several views of the drawings.

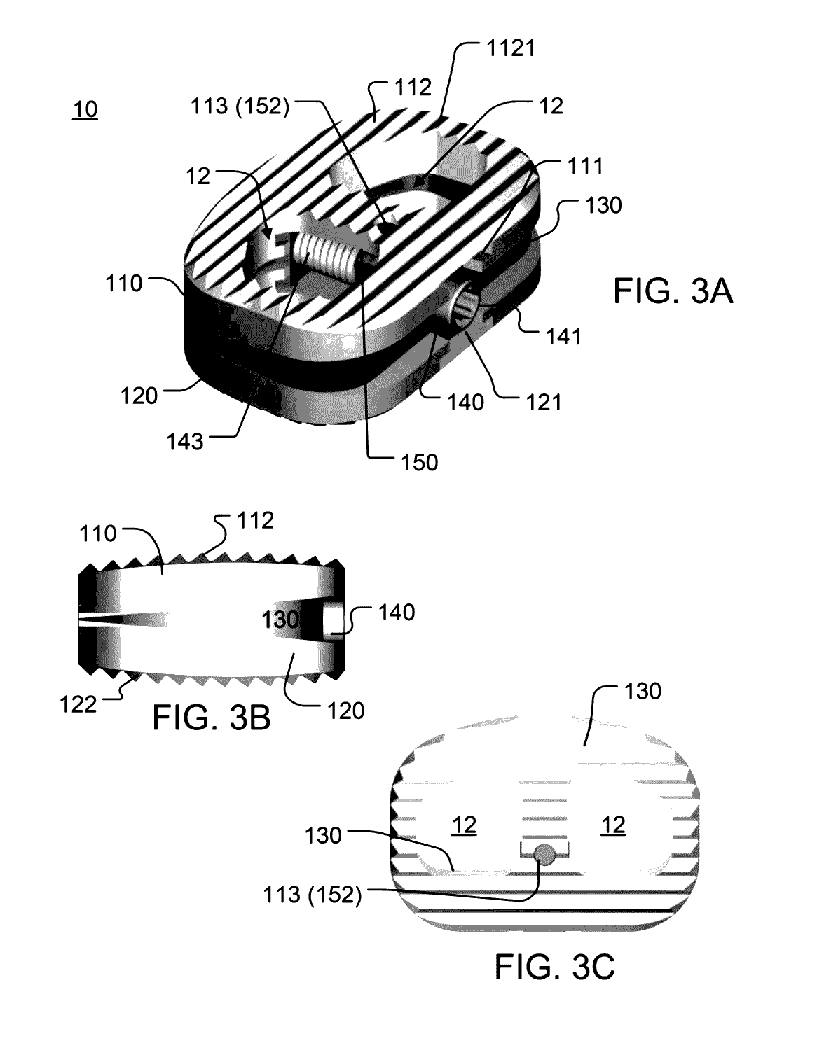

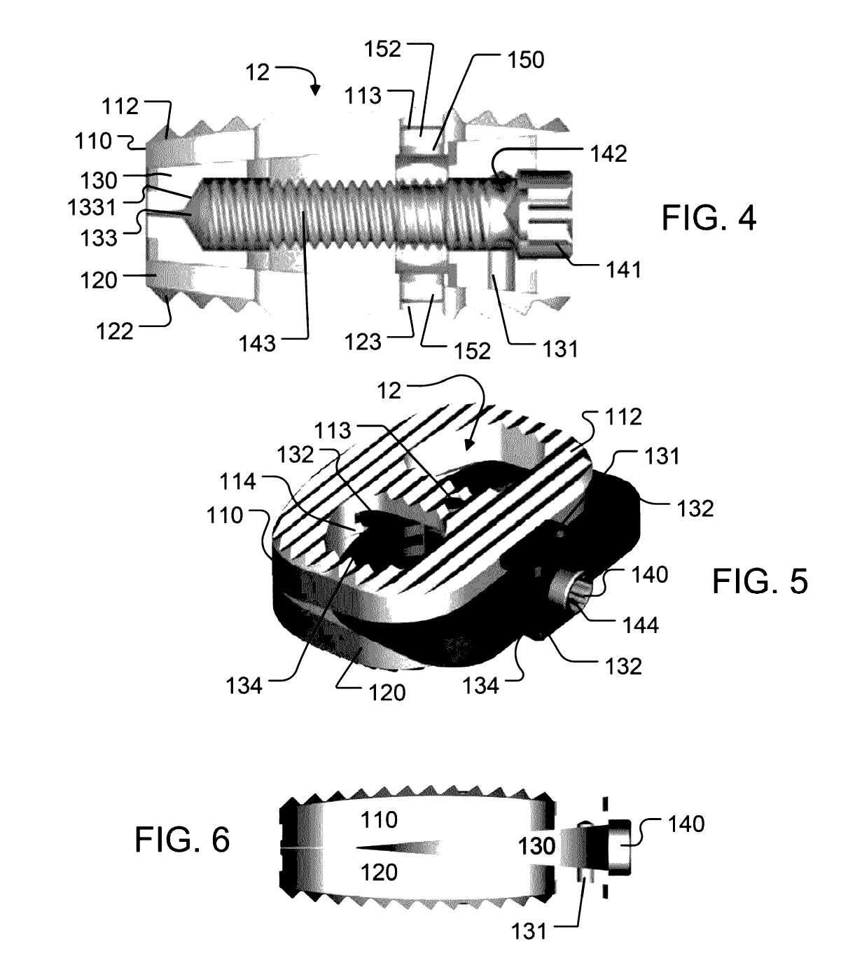

FIGS. 3A-3C illustrate perspective, side, and top (or bottom) views, respectively, of a cage 10; FIG. 4 illustrates a side cross-section view of the expandable cage 10; and FIGS. 5-6 illustrate perspective and side views, respectively, of the cage 10 with an intermediary plate 130 partially removed from (or installed in) the cage 10. The cage 10 comprises a self-distracting expandable cage.

Referring to FIGS. 3A-6, the cage 10 includes a first plate 110 and a second plate 120, which together form a plate system 110/120, the intermediary plate 130, an actuator 140 and an anchor 150. The cage 10 further includes at least one graft chamber 12 to hold a bone graft. The plate system 110/120 is configured to receive and guide the intermediary plate 130 as it travels between the plates 110, 120.

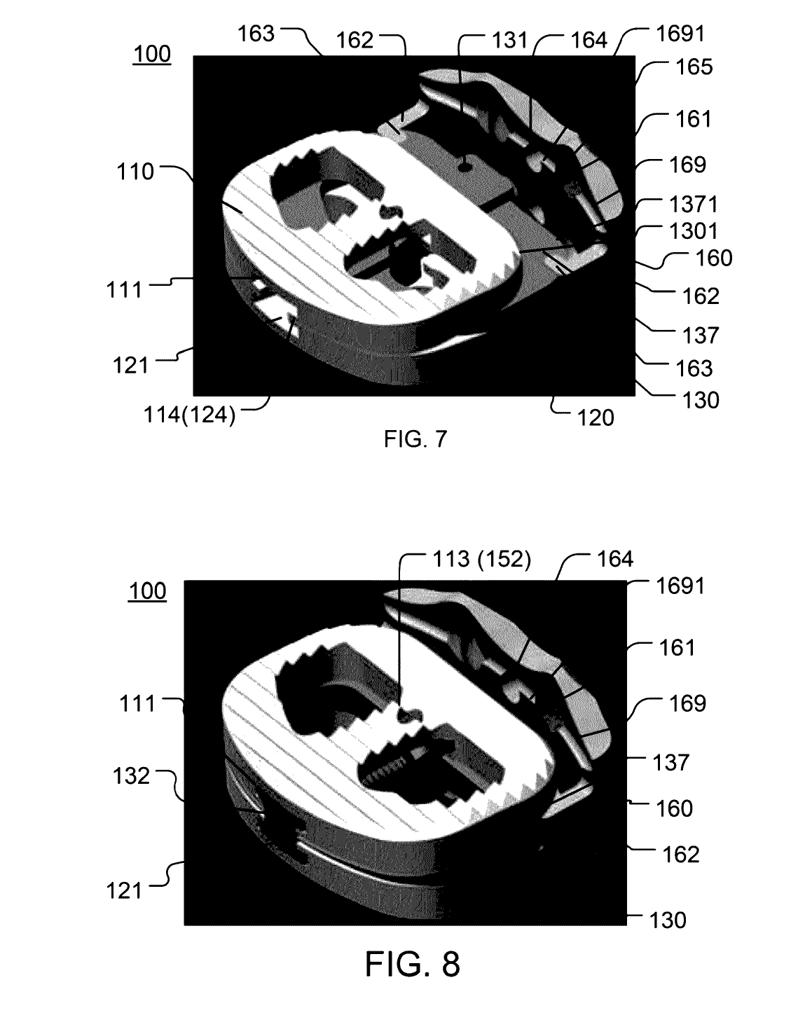

The first plate 110 has an outer surface 112 that may include a plurality of bone interface members 1121, such as, for example, teeth, serrations, protrusions (e.g., triangular, pyramidal, conical, semi spherical, rectangular, cylindrical, diamond, elliptical, and/or irregular shapes, or the like). The inner surface (not shown) of the first plate 110 may be substantially smooth to provide a low-friction interface with a surface 1301 (shown in FIG. 7) of the intermediary plate 130. The bone interface members 1121 engage with the bony surface of vertebral bodies in or near the treated area. The bone interface members 1121 may be formed integrally with the surface 112 and may vary in profile, distribution, size, and number. The configuration of the surface 112, including bone interface members 1121, should be sufficient to securely hold the cage 10 in the treated area after surgery while the treated area heals and undergoes fusion.

The second plate 120 has an outer surface 122, which may be configured substantially the same as the surface 112 on the first plate 110. The surface 122 may have a configuration that differs from that of surface 112. The surface 122 may include a plurality of bone interface members 1121. The inner surface (not shown) of the second plate 120 may be substantially smooth to provide a low-friction interface with the surface of the intermediary plate 130, which is opposite to the surface 1301 shown in FIG. 7.

The first and second plates 110, 120 include guide tracks 111, 121, respectively, that are located on (or in) the inner surfaces of the plates. The guide tracks 111, 121 may each have a T-shape, with a narrower guide interface 114, 124, respectively (shown in FIG. 7). The guide interfaces 114, 124 may function to slidably hold the corresponding guides 132 in the guide tracks 111, 121, respectively. When the plates 110, 120 are superimposed, as seen in FIGS. 3A-6, the guide tracks 111, 121 may align to form an I-shape opening (shown in FIG. 7).

The guide tracks 111, 121 engage corresponding guides 132 (shown in FIG. 5) on the intermediary plate 130 to guide the intermediary plate 130 as it travels in (or out from) the plate system 110/120. As seen in FIG. 5, each guide 132 may have a guide channel 134 to form a T-shape, and the combination of the upper and lower guides 132 (shown in FIG. 5) may collectively form an I-shape. The guide tracks 111, 121 engage the guides 132 to securely couple the first and second plates 110, 120 to each other, and to the intermediary plate 130, so that the intermediary plate 130 may move in or out of the space formed between the first and second plates 110, 120 along the longitudinal axis of the guide tracks 111, 121.

Referring to FIG. 4, the first plate 110 may include a receiver 113 that is configured to receive and hold an anchor lock 152. The receiver 113 may be formed as an opening (e.g., a hole) that passes from the inner plate surface (opposite surface 112) and through the thickness of the plate 110 and surface 112, as seen in FIG. 4. Alternatively, the receiver 113 may be formed as a notch or recess (not shown) in the inner surface of the plate 110 configured to receive the anchor lock 152.

The second plate 120 may include a receiver 123 for another anchor lock 152, as seen in FIG. 4. The receiver 123 may be substantially the same as the receiver 113. The receivers 113 and 123 may be aligned so as to ensure proper alignment of the first and second plates 110, 120 when the plates are superimposed one on top of the other, as seen in FIG. 4. The receivers 113, 123 may function jointly to secure the anchor 150 in the cage 10, preventing the anchor 150 from rotating and/or moving with respect to the plate system 110/120.

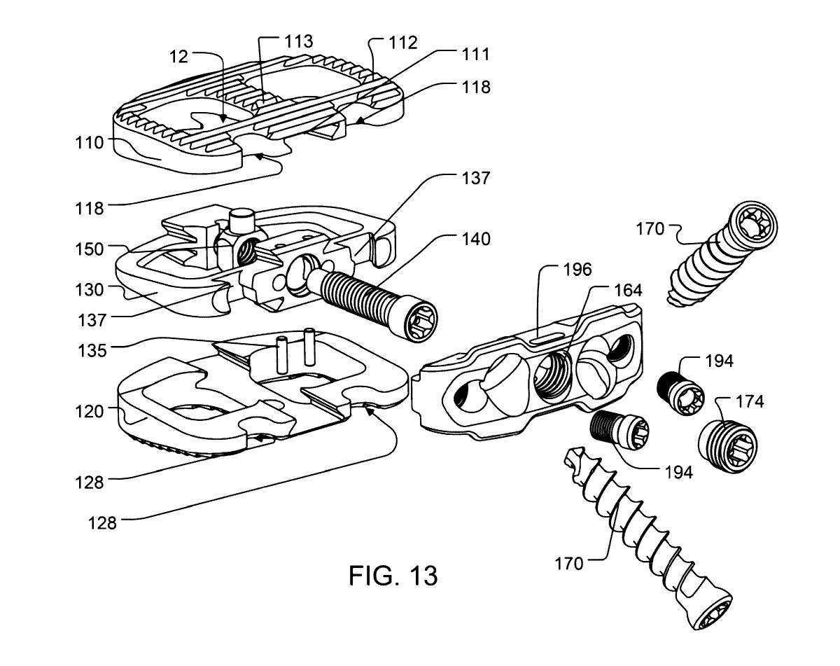

The intermediary plate 130 has an intermediary plate body with openings that, together with corresponding openings in the plate system 110/120, form the graft chamber(s) 12. The intermediary plate body includes an actuator channel 133 that is configured to receive and hold the actuator 140 within the intermediary plate body, as seen in FIG. 4. The actuator channel 133 may include a wall 1331 at a posterior end of the intermediary plate body 130 (shown in FIG. 4). The wall 1331 may be configured to contact and receive a lateral force from the actuator 140 to move the intermediary plate 130 into the plate system 110, 120. The intermediary plate 130 may include one or more pin receptacles 131 that receive corresponding pins 135 (shown in FIG. 13) to secure the actuator 140 with respect to the intermediary plate 130, allowing the actuator 140 to rotate about its longitudinal axis, but holding it securely in place along the longitudinal axis. As seen in FIGS. 4 and 5, the intermediary plate 130 may include an upper and a lower pin receptacle 131, so as to secure the actuator 140 therebetween. The pins 135 may be configured to fit in the smaller-diameter areas adjacent an actuator neck 142, which is located between the larger-diameter areas of an actuator body 143 and actuator head 141, thereby securing the actuator 141 with respect to the pins 135 along the longitudinal axis of the actuator 140, so as to prevent the actuator 140 from moving longitudinally (i.e., along the longitudinal axis of the actuator 140) with respect to the pins 135, but allowing the actuator 140 to rotate about its longitudinal axis. The pins 135 (shown in FIG. 13) are configured to receive a lateral force from the actuator 140 to move the intermediary plate 130 into or out of the plate system 110, 120.

The actuator 140 may be a screw, a bolt, or the like. As seen in FIG. 4, the actuator 140 may include the actuator head 141, the actuator neck 142, and the actuator body 143. The actuator head 141 may include a driver interface 144 (shown in FIG. 5) that is configured to receive and be engaged by a tool (such as, for example, a polyaxial screw driver, not shown) to rotate with respect to the anchor 150. The actuator neck 142 may have a smaller diameter than the diameters of the actuator head 141 or the actuator body 143, so as to receive and engage the pins 135 (shown in FIG. 13) that may be placed in the pin receptacles 131 of the intermediary plate 130, thereby conveying and applying a force to and driving the pins together with the actuator neck 142 as the actuator 140 moves along its longitudinal axis in the spacer 10. The actuator body 143 may include a shaft having, for example, a threading that is configured to engage a corresponding threading in the anchor 150.

The anchor 150 may include, for example, an anchor nut, which may include a pair of opposing anchor locks 152, as seen in FIG. 4, to prevent the anchor nut from rotating when the anchor locks 152 are seated in the receivers 113, 123 of the plate system 110/120. As seen in FIG. 4, the anchor locks 152 may include male protrusions that are configured to fit in the receivers 113, 123 and keep the anchor 150 from moving (e.g., rotating). Alternatively, the anchor locks 152 may include female recesses (not shown) that are configured to receive male protrusions (not shown) that may be provided instead of the receivers 113, 123.

The anchor 150 may include a threading (not shown) that may engage a corresponding threading on the actuator body 143, so as to drive the intermediary plate 130 in or out from the plate system 110/120 when the actuator 140 is manipulated. In the case where the actuator 140 is a screw or a bolt, turning of the actuator head 141 in a first direction will cause the actuator body 143 to advance with respect to the anchor 150, thereby transferring and applying a force in a posterior direction against the channel wall 1331 (shown in FIG. 4) at the posterior end of the intermediary plate body 130 and the pins 135 (shown in FIG. 13) at the anterior end of the intermediary plate body, forcing the intermediary plate 130 to move toward the posterior end of the plate system 110/120. Turning of the actuator head 141 in a second direction (opposite to the first direction) will cause the actuator body 143 to withdraw with respect to the anchor 150, thereby transferring and applying a force in an anterior direction against the pins 135 (shown in FIG. 13) and forcing the intermediary plate 130 to move away from the posterior end of the plate system 110/120.

The cage 10 may be configured to expand in height as the intermediary plate 130 is driven deeper into the plate system 110/120. The cage 10 may be constructed in different sizes and shapes to properly match patient anatomy. For instance, the height, width and depth of the cage 10 may be constructed to match the space occupied by, for example, the intervertebral disc that is to be replaced. For instance, the cage 10 may be constructed to restore a space between adjacent vertebrae that may span from, for example, a height of less than 10.4 mm to a height of greater than 20.1 mm. As illustrative, non-limiting examples, the cage 10 may have a height that expands from, for example, about 10.4 mm to about 11.7 mm; about 11.0 mm to about 12.3 mm; about 12.1 mm to about 14 mm; about 13.0 mm to about 15 mm; about 14.1 mm to about 16.5 mm; about 15.7 mm to about 18.1 mm; about 17.7 mm to about 20.1 mm; or the like. The height of the cage 10 may vary as a function of the insertion length of the intermediary plate 130 in the plate system 110/120, so that when the intermediary plate 130 is driven deeper into the plate system 110/120, the height of the cage 10, including the plate system 110/120 will increase.

The cage 10 is shown in FIGS. 3A-25 as having a substantially closed self-distracting design. It is noted that the cage 10 may have an open design (e.g., U-shape, fork-shape, or the like), or any combination of closed and/or open designs. The cage 10 may be configured as non-self-distracting, as will be understood by those skilled in the art. In the case of an open design (not shown), at least one graft chamber may be formed by the inner walls of the cage and an inner wall of a plating device, such that when the plating device is removed, the graft chamber is open. An example of a U-shaped or fork-shaped open cage is illustrated in FIGS. 33-34. An example of a combination closed and open design of a cage is illustrated in FIGS. 35-36. The cage 10 may be used alone or with a plating device.

The cage 10 may be configured to interchangeably mate with different interlocking plating devices, such as, for example, plating devices 160, 190, 196, described below. The lower profile plating devices (such as those having one or two bone fastener openings—for example, plating device 196) may be mated to a lower-height cage 10. The higher profile plating devices (such as those having three, four, or more bone fastener openings—for example, plating device 190) may be mated to a larger-height cage 10. The plating devices described herein may be configured to attach to adjacent vertebrae intradiscally (i.e., the plating device is designed to fit completely within the space provided between adjacent vertebrae), extradiscally (i.e., the plating device is designed to attach to an outer surface of one or both adjacent vertebrae), or interdiscally (i.e., the plating device is designed to have a portion that completely fits within the space provided between adjacent vertebrae and has a portion that attaches to an outer surface of one or both adjacent vertebrae).

Various arrangements of the cage, plating devices and/or bone fasteners disclosed herein may include one or more features configured to facilitate sagittal and/or coronal visibility. For example, the cage and/or plating device may comprise a radiopaque material visible via x-ray or similar forms of imaging modalities. As such, the structures may enable accurate positioning and/or placement of the cage system within and/or along spinal column.

FIGS. 7-8 illustrate perspective views of the cage 10 with an exemplary plating device 160 coupled to the intermediary plate 130 of the cage 10. FIG. 7 shows the plating device 160 with intermediary plate 130 partially removed from (or installed in) the plate system 110/120; and, FIG. 8 shows the plating device 160 with intermediary plate 130 substantially completely installed in the plate system 110/120. As seen in FIGS. 7-8, the intermediary plate 130 may include a plate connector interface(s) 137 at its end(s) to receive corresponding intermediary plate connector interfaces 163 on the plating device 160. The intermediary plate body may be configured to include plate set fastener contact portions 1371 located between the plate connector interfaces 137 and the inner face 165 of the plating device 160 when the plating device 160 is coupled to the intermediary plate 130. Each plate set contact portion 1371 may be sandwiched between the respective intermediary plate connector interface 163 and inner face 165 of the plating device 160, and securely squeezed therebetween by a respective plate set fastener 194, which contacts a wall of the plate set contact portion 1371 and applies a force toward the intermediary plate connector interface 163 to securely fasten the plating device 160 to the intermediary plate 130.

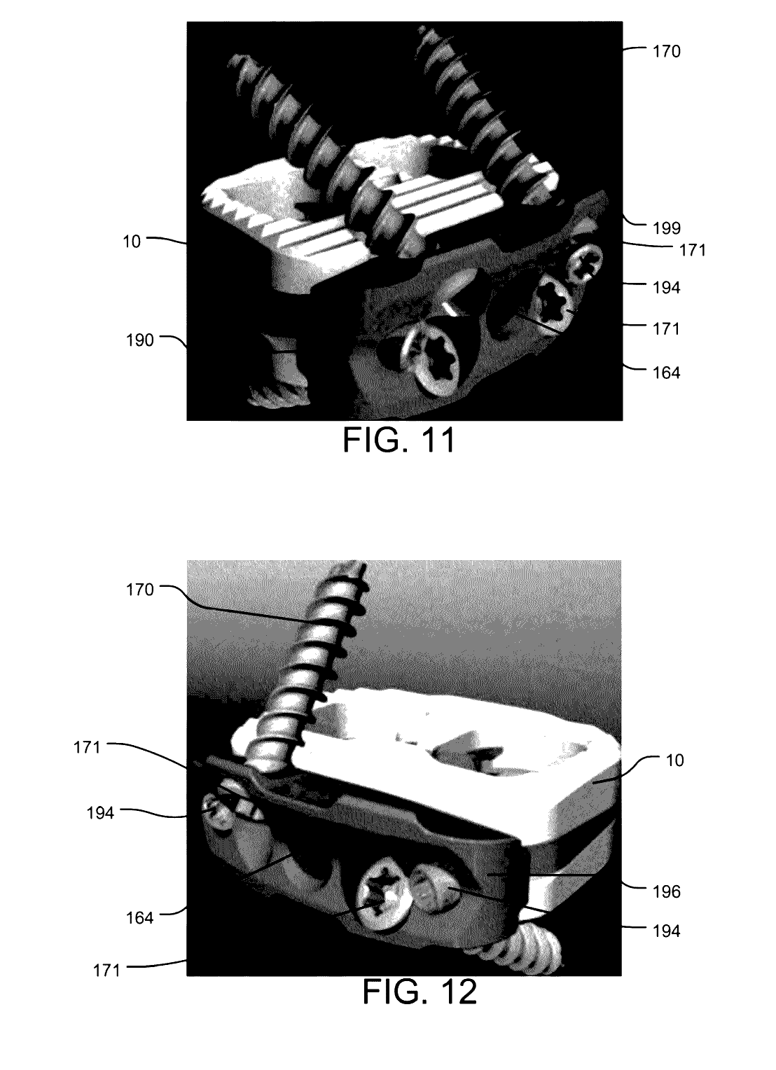

As seen in FIGS. 7-8, the plating device 160 includes a plurality (e.g., two, three, four, or more) of bone fastener apertures 161 that receive corresponding bone fasteners 170 (shown in FIG. 11) to securely attach the plating device 160 to adjacent vertebrae. The plating device 160 may include the intermediary plate connectors 162 and intermediary plate connector interfaces 163 to attach the plating device 160 to the intermediary plate 130. The plating device 160 includes a driver aperture 164 that allows a tool end (not shown) to pass through the plating device 160 and engage the actuator 140 to drive the intermediary plate 130 into (or out from) the plate system 110/120. The plating device 160 includes one or more bone interfaces 169 (e.g., upper and lower bone interfaces), each of which contacts a portion of the adjacent vertebrae during implanting. The bone interfaces 169 may include a lip portion 1691 (as seen in FIGS. 7-8) to assist in aligning the plating device 160 with respect to the adjacent vertebrae. The distance between the upper and lower lip portions 1691 may be selected to be substantially the same as the desired height of the cage 10.

The bone fastener(s) 170 may include, for example, multi-purpose bone screws. The bone fastener(s) 170 may include a head portion 171, a neck portion, and a shaft portion. The bone fastener(s) 170 may be configured at its distal end to penetrate and facilitate insertion of the bone fastener 170 into bone. At its proximal end, the head portion may have a substantially spherical shape. The shaft portion may have a thread that is adapted to be screwed into a bone, such as, for example, a vertebra. Alternative formations may be formed in/on the shaft portion which provide the intended purposes of securing the bone fastener 170 within a bone, as described herein. The shaft portion may have a tapered shape, which may be provided with a high pitch thread. It is noted that the length, diameter, thread pitch, and thread diameter ratio of the shaft portion may be selected based on the particular application of the bone fastener 170, as understood by those skilled in the art. The bone fastener 170 may include a self-drilling tip, a serrated threaded flute, a hexalobular drive, or the like.

The bone fastener head portion 171 may include a tool receptacle 172 at its proximal end that is configured to receive a driver tool (not shown) to, e.g., drive the fastener 170 into bone. The tool receptacle 172 may have a hexagon shape, a torque-screw shape, or any other shape that may facilitate the bone fastener 170 being driven into a bone by the driver tool.

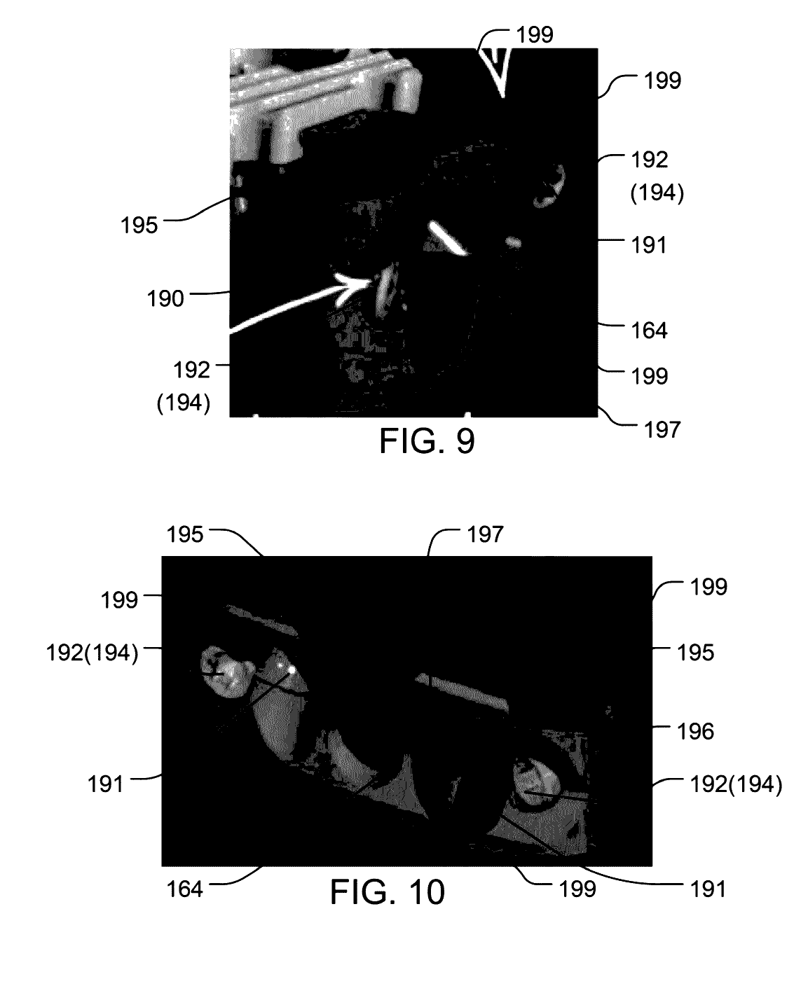

FIG. 9 illustrates a perspective view of a plating device 190 that may be used with the cage 10. As seen, the plating device 190 includes one or more bone fastener apertures 191 (e.g. four openings), one or more plate set fastener apertures 192 (e.g., two openings), a driver tool aperture 164, one or more intermediary plate connectors 195 (e.g., two), and one or more bone interfaces 199. The bone fastener aperture 191 may include a flange 197 that is contacted and forced by the head portion 171 of a bone fastener 170 to secure the plating device 160 to a vertebra. The plate set fastener aperture 192 may receive a corresponding plate set fastener 194 to securely affix the plating device 190 to the intermediary plate 130. The intermediary plate connector 195 may have an L-shape, as seen in FIG. 9, or any other shape as understood by those skilled in the art, without departing from the scope or spirit of the disclosure. The bone interface 199 may be configured to contact and rest against an edge portion of a vertebra (e.g., an upper and/or lower edge portion of the vertebra) during implanting. The bone interface 199 may be configured to assist in properly seating the plating device 190 with respect to adjacent vertebrae, and securing the plating device 190 to the vertebrae.

FIG. 10 illustrates another example of a plating device 196 that may be coupled to the cage 10. The plating device 196 may have a structure similar to that of the plating device 190, except that it has two bone fastener apertures 191 compared to the four bone fastener apertures 191 in the plating device 190 (shown in FIG. 9), thereby providing a lower-profile configuration.

FIGS. 11 and 12 illustrate attachment of the plating devices 190 (shown in FIG. 9) and 196 (shown in FIG. 10) to the cage 10 and provided with bone fasteners 170 and plate set fasteners 194.

FIG. 13 illustrates an exploded view of the plating device 196 (shown in FIG. 10) and cage 10 provided with bone fasteners 170. As seen, the first plate 110 may be provided with bone fastener passageways (or cutouts) 118 to allow passage of the bone fasteners 170, so as to provide a more compact design. Similarly, the second plate 120 may be provided with bone fastener passageways 128 to allow passage of the bone fasteners 170 for a more compact design. The cage 10 comprises the plate system 110/120 with intermediary plate 130 placed therebetween and movably secured to the plate system 110/120 by the actuator 140, anchor 150 and pins 135. The plating device 196 may be secured to the intermediary plate body 130 by means of the intermediary plate connectors 195 (shown in FIG. 10), plate connector interfaces 137 and plate set fasteners 194.

The plate set fastener(s) 194 may include a head portion, a neck portion and a shaft portion, as seen in FIG. 13. The plate set fastener(s) 194 may include a bolt, a screw, a nut, or the like. The plate set fastener(s) 194 may include a head portion with a tool receiver to turn and drive a plate set fastener shaft 1941 (e.g., shown in FIG. 28) of the plate set fastener(s) 194 using, for example, a driver tool (not shown).

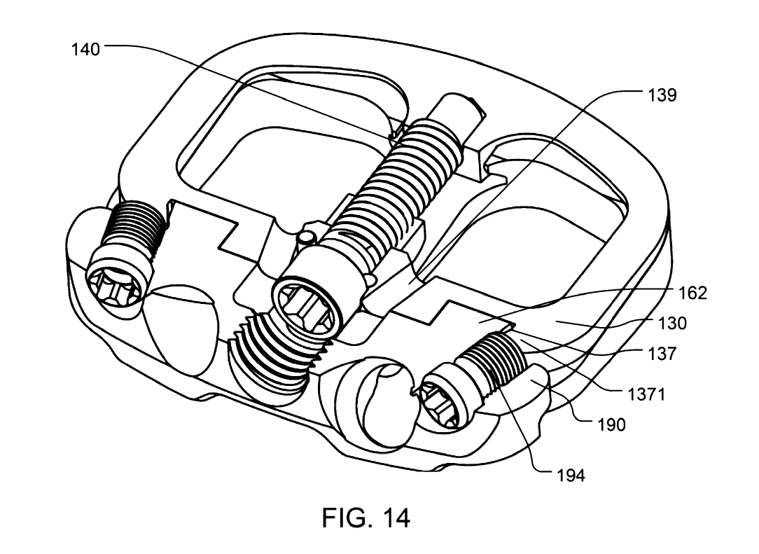

FIG. 14 illustrates a partial cut-away view of the plating device 196 and cage 10, provided with one or more channels 139 for introduction of, for example, putty style graft material into the cage 10 after the cage 10 is installed. The plating device 196 and cage 10 may include one or more channels 139 that exit in the plates 110, 120.

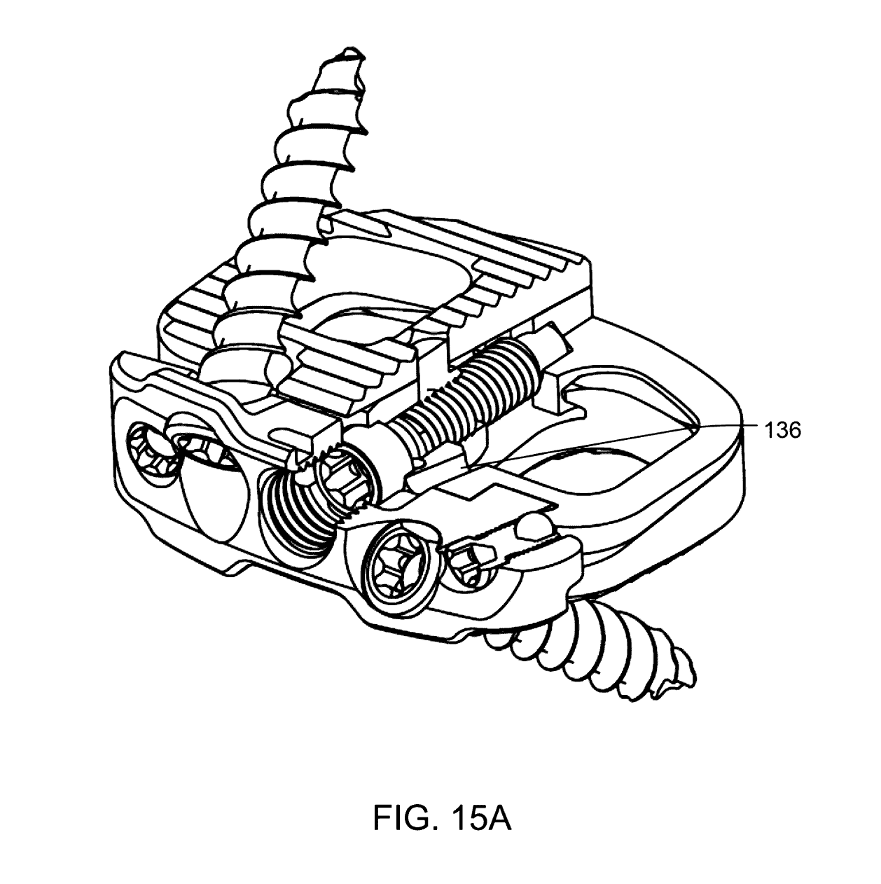

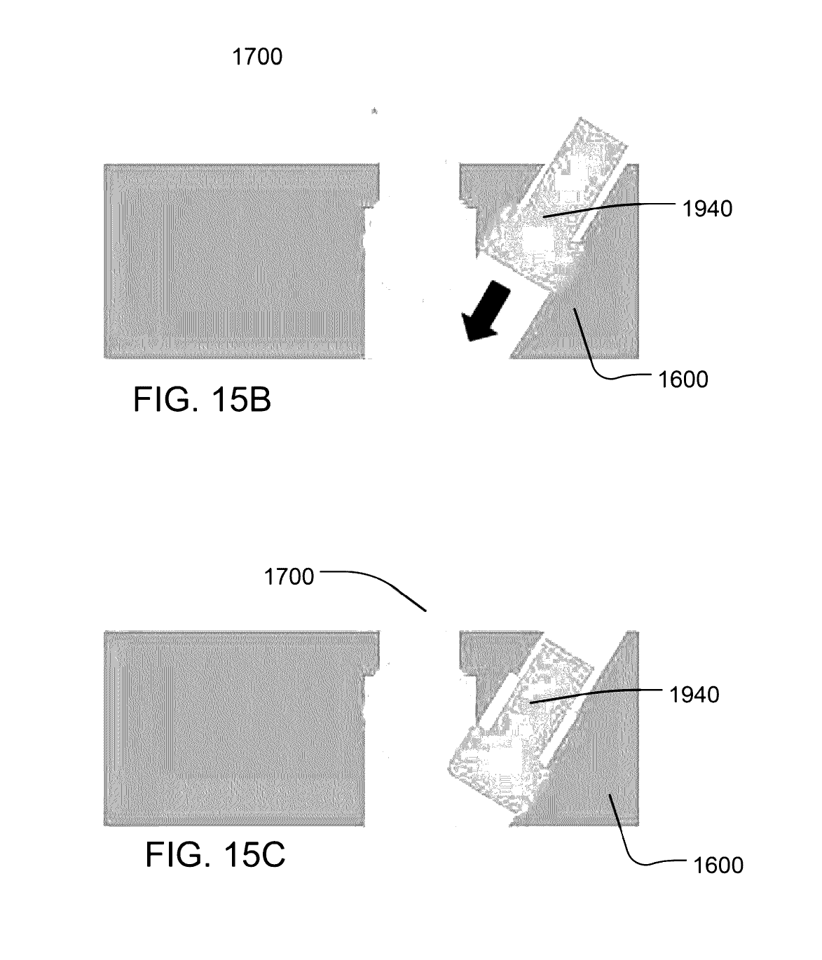

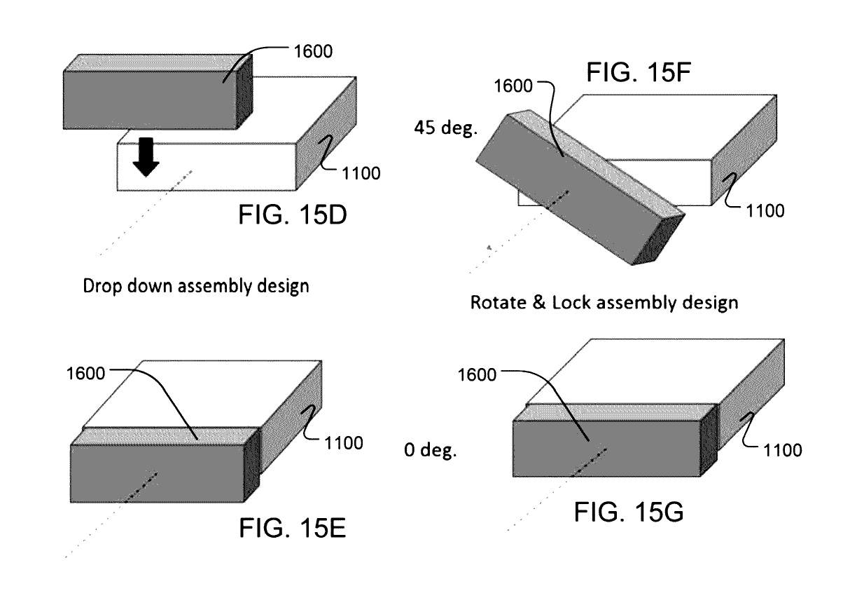

FIG. 15A illustrates a partial cut-away view of the plating device 196 and cage 10 similar to that of FIG. 14, except that the one or more channels 136 exit into the graft chamber(s) 12. Putty style graft material, for example, may be injected into the graft chamber(s) 12 via channel(s) 136 after the cage 10 is, for example, inserted to its proper location and expanded to its proper height.

FIGS. 15B and 15C illustrate an example of a fastener blocking mechanism that may be included in the plating device 1600 (or 160 or 190 or 196 or 1601). The fastener blocking mechanism may include a plate set fastener 1940 (or 194) that may be positioned as seen in FIG. 15B to allow for passage of a bone fastener 1700 (or 170). After the bone fastener 1700 is installed, the plate set fastener 1940 may be backed out to block the bone fastener 1700 from backing out, as seen in FIG. 15C.

FIGS. 15D and 15E illustrate an example of a drop-down assembly design that may be implemented for the plating device 1600 and cage 1100 (or 10). In this example, the plating device 1600 may be dropped down (or pulled up) to engage with the cage 1100.

FIGS. 15F and 15G illustrate an example of a rotate and lock assembly design that may be implemented for the plating device 1600 and cage 1100. In this example, the plating device 1600 and cage 1100 are configured to rotate and lock. The assembly may start at about 45 degree plate offset and then, after rotation to 0 degree, the plating device 1600 may lock and align with the cage 1100. The plating device 1600 (or 160, or 190, or 196, or 1601) may include the intermediary plate connector(s) 195 (shown in FIGS. 9 and 10) having a male L-shape that allow for rotation and locking of the plating device 1600 to the cage 1100. As seen in FIG. 9, the cage 1100 (or 10) may include a corresponding plate connector interface(s) that receives and alignably engages the intermediary plate connector(s) when the plating device 1600 is rotated and locked into position (shown in FIG. 15G).

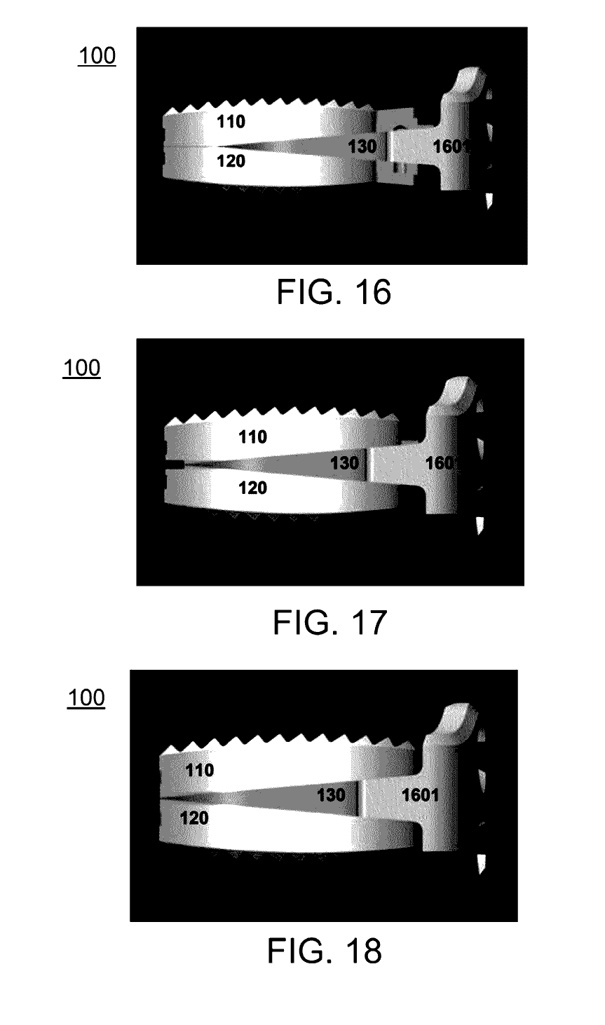

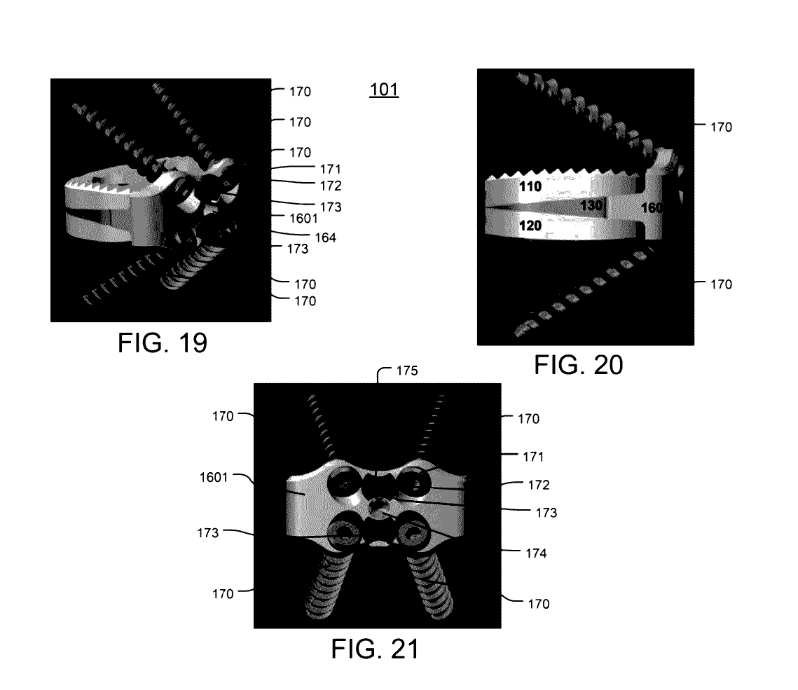

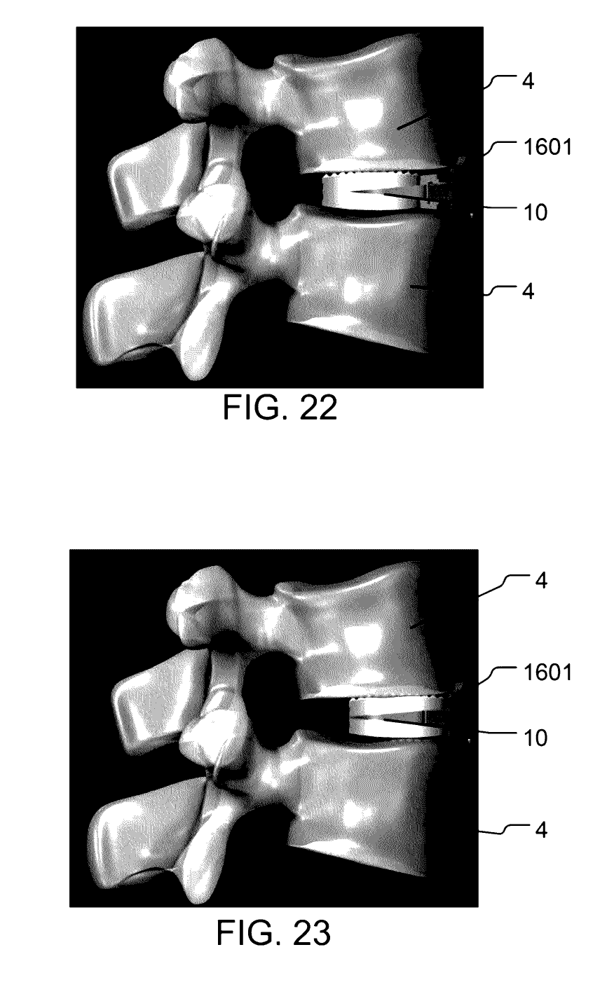

FIGS. 16-25 illustrate various stages of installing the cage 10 with plating device 160 in a patient. More specifically, FIGS. 16-18 show insertion of the intermediary plate 130 into the plate system 110/120 at three different stages, with the intermediary plate 130 being securely fastened to a plating device 1601; FIGS. 19-21 show substantially complete insertion of the intermediary plate 130 in the plate system 110/120 with bone fasteners 170 installed through corresponding bone fastener apertures 191 in the plating device 1601; and FIGS. 22-25 illustrate installation of the cage 10 with plating device 1601 in between a pair of adjacent vertebrae 4.

Referring to FIGS. 16-18 and 22-23 simultaneously, the cage 10 and plating device 1601 (together forming a cage system 100) may be configured for use in, for example, anterior approach and discectomy applications. For instance, after a surgical area is cleaned on a patient, an incision made, muscle tissue and/or organs moved to the side(s), and other common surgical procedures carried out, a disc may be incised, removed, and the space prepared for implanting of a cage system. The bone surfaces and edges on the adjacent vertebrae may be carefully contoured, as appropriate.

Following a discectomy procedure, a medical professional may determine an appropriate size of the cage system 100 by selecting an appropriately dimensioned cage 10 and an appropriately dimensioned plating device 1601, which may be selectable based on, for example, height, width, depth, number of graft chambers, configuration of graft chambers, configuration of outer surface 112 (including bone interface members 1121), and the like. Upon selecting the appropriate cage 10 and plating device 1601, one or more of an ACIF, ALIF, or the like may be performed by placing the cage system 100 between adjacent vertebrae 4 in the space formed by the removed degenerated disc (shown in FIGS. 22-23). Placement of the cage system 100 within spinal column may prevent spaces between adjacent vertebrae 4 from collapsing, thereby preventing adjacent vertebrae from resting immediately on top of one another and inducing fracture of vertebra 4, impingement of the spinal cord, and/or pain. Additionally, such cage systems 100 may facilitate fusion (e.g., bone to grow together) between adjacent vertebrae 4 by stabilizing adjacent vertebrae 4 relative to one another.

Referring to FIGS. 16 and 22, the cage 10 may be placed slightly deeper than normal into the space between the vertebrae 4. Then, as seen in FIGS. 17-18, and 23, the actuator 140 (shown in FIG. 3A) may be turned by a driver tool (not shown), causing the cage 10 to expand and drawing the cage toward the anterior face of the vertebrae 4 until it reaches the position seen, for example, in FIGS. 18 and 23. The driver tool may engage and turn the actuator 140 via the driver aperture 164. In this regard, one or more portions (e.g., bone interfaces) of the posterior face of the plating device 1601 may be seated against the surfaces of the adjacent vertebrae 4.

If the plating device 1601 includes one or more bone interfaces (e.g., bone interface 199, shown in FIGS. 9-10), a portion of the plating device 1601 may be positioned in the intervertebral disc space. Alternatively (or additionally), one or more portions of the plating device 1601 may be positioned externally and against corresponding surface portions of the vertebra(e) 4, in contact with the surface(s) of the vertebra(e) 4 so as to provide a snug and secure fit to the vertebrae 4.

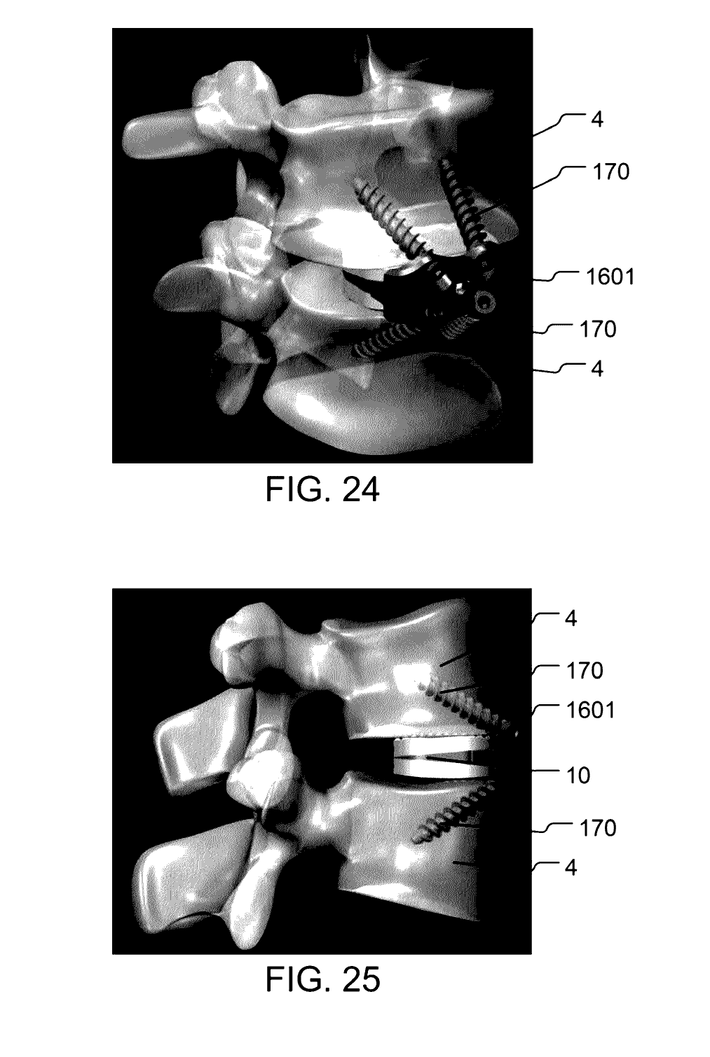

Once the cage 10 and plating device 1601 are properly installed with respect to the vertebrae (e.g., as seen in FIG. 23), a medical professional may prepare one or more bores or holes in the vertebra 4 intended to receive bone fasteners 170 (shown in FIGS. 24-25). In this regard, hard bone surface may be removed and a guide track may be inserted under x-ray guidance into the vertebrae 4. The depth and position of the guide track may be checked. Where the bone fastener 170 includes a bone screw, a thread may be tapped into the bone to form a tap (not shown) to receive and securely hold the bone fastener 170. The process would be repeated for each bone fastener 170. Such holes may be formed with the aid of a separate drill guide (not shown) positioned proximate or abutting vertebra 4 and inserting a drill therethrough. Alternatively, such holes may be formed free hand, without the use of a drill guide.

After the cage 10 and plating device 1601 are properly installed with respect to the vertebrae 4 (e.g., as shown in FIG. 23), the bone fastener(s) 170 may be installed. In this regard, a driver tool (not shown), as is known by those skilled in the art, may be used to turn and drive the bone fastener(s) 170 into the vertebrae 4. It is noted that the bone fastener(s) 170 may be aligned with the tap (not shown) in the bone and screwed into the threaded tap.

Alternatively, the bone fasteners 170 may be partially installed in the tap before being contacted by the driver tool. Once the bone fasteners 170 are implanted in the desired position, the driver tool may be removed and the process repeated for each bone fastener 170.

Since the spinal column is subject to dynamic forces, often changing with each slight movement of the patient, such bone fasteners 170 could have a tendency to back out (e.g., unscrew) and/or dislodge from the cage system 100, thereby limiting the cage system's 100 ability to stabilize adjacent vertebrae 4, and consequently, promote fusion. Additionally, if bone fasteners 170 back out and/or dislodge from the cage system 100, they may inadvertently contact, damage, and/or irritate surrounding tissue.

The cage system 100 may include one or more bone fastener locks 173, as shown in FIGS. 19 and 21. In this regard, the plating device 1601 may have a face that defines one or more apertures and includes a corresponding bone fastener lock 173 (e.g., any screw blocking mechanism). The bone fastener lock 173 may include a blocking element cutout 175 that is configured to substantially match the other diameter of the bone fastener head 171 to allow the bone fastener head 171 to pass the bone fastener lock 173 unobstructed during installation. Once the bone fastener head 171 is seated properly and securely, the bone fastener lock 173 may be rotated so as to block a portion of the bone fastener head 171, as seen in FIG. 21, thereby preventing the bone fastener 170 from withdrawing, unscrewing, or otherwise being removed from the implant system 100. The cage system 100 may further include a blocking element 174 (shown in FIG. 21), that may be installed (e.g., screwed, snapped into, or the like) in the driver tool aperture 164 to engage the blocking element cutouts 175 and prevent the bone fastener locks 173 from turning to an unlocked position.

The bone fastener lock 173 may include, for example, the offsetting element 24 (and associated structures), or other bone screw locking structures described in U.S. patent application Ser. No. 14/956,084, filed Dec. 1, 2015, titled “INTERVERTEBRAL IMPLANTS AND RELATED SYSTEMS AND METHODS,” the descriptions of which are incorporated herein by reference in the entirety, as if fully set forth herein.

As discussed above, the graft chamber(s) 12 (e.g., shown in FIG. 3A, 3C, 4-5, or 7-8) and/or channel(s) 139 (shown in FIG. 14), 136 (shown in FIG. 15) may be filled with a radiolucent material such as tissue grafts. For instance, the graft chamber(s) 12 may be packed with bone graft, and the one or more channels 139 (or 136) may be filled with, for example, putty style graft material. Bone graft material may facilitate bone and tissue ingrowth in and through the cage system 100. Accordingly, bone graft may promote fusion, i.e., the joining of two or more vertebrae 4.

The cage system 100, including the cage 10 and plating device 1601, may be configured such that bone graft material packed within cage system 100 may be retained therein. That is, interior surface(s) of the cage 10 and plating device 1601 may define one or more non-uniform or uneven surfaces which, upon receipt of packed bone graft material, may act to hold bone graft material therein.

After the bone graft materials are installed, and the bone fasteners 170 are securely and properly placed in corresponding taps, and the installation of the cage system 100 completed, the area may be cleaned, checked, closed and other post-operative procedures carried out, as is known in the art.

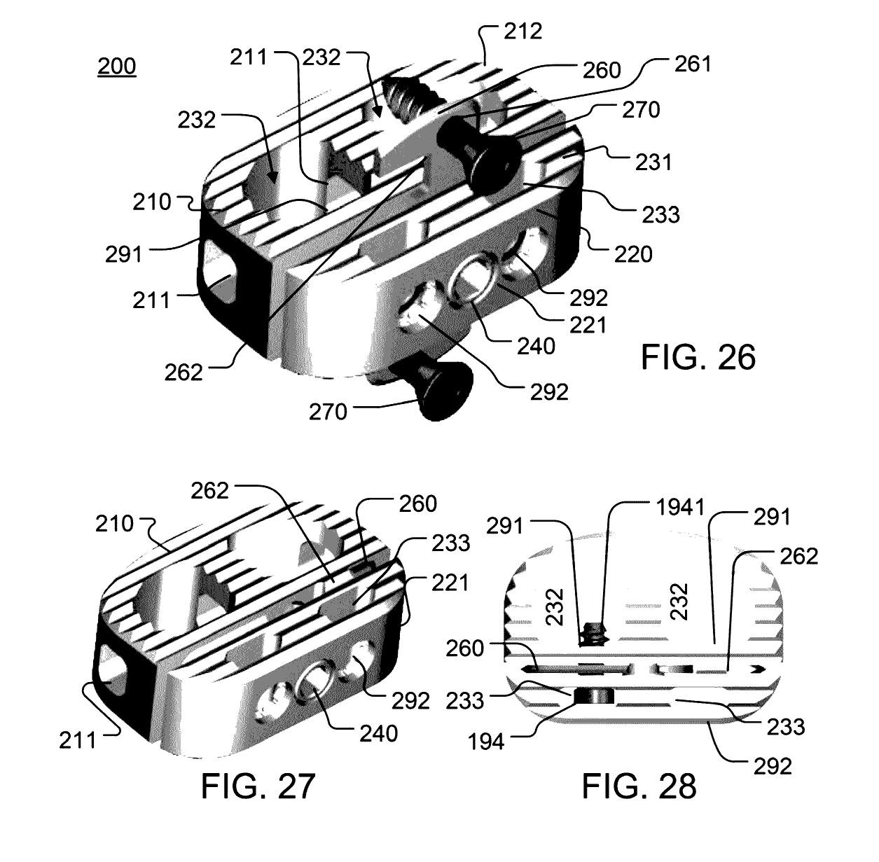

FIGS. 26-28 illustrate perspective and top (or bottom) views of an example of a cage system 200 that is constructed according to the principles of the disclosure. The cage system 200 includes a posterior plate 210 and an anterior plate 220. The posterior plate 210 may be removably coupled to the anterior plate 220 by means of an actuator 240 and/or one or more plate set fasteners 194. The cage system 200 may include an anchoring plate 260.

The actuator 240 may include, for example, a bolt, a screw, a pin, a lever, or the like. The actuator 240 may be configured to fasten the anterior plate 220 to the posterior plate 210 while simultaneously being operable to rotate the anchoring plate 260 from a retracted position (shown in FIG. 27) to an engaged position (shown in FIG. 26), or from the engaged position to the retracted position. Alternatively, the actuator 240 may be coupled to only one of the anterior plate 220 or the posterior plate 210 and configured to rotate the anchoring plate 260 from the retracted (or engaged) position to the engaged (or retracted) position. In the latter instance, the anterior plate 220 may be held fastened to the posterior plate 210 by means of one or more of the plate set fasteners 194.

The posterior plate 210 may include one or more posterior graft chambers 232 that are formed by walls that may include one or more apertures (or windows) 211. The non-limiting example of the posterior plate 210 includes two graft chambers 232 with apertures (or windows) 211 formed in the wall between the chambers 232, and apertures (or windows) 211 formed in the walls between the chambers 232 and outside of the posterior plate 210. The posterior plate 210 further includes surfaces 212, which may be similar to the surfaces 112, shown in FIG. 3B. The posterior plate 210 may include one or more coupler apertures 291 that may be aligned with corresponding, respective coupler apertures 292 in the anterior plate 220. The coupler aperture(s) 291 may include a threading that is configured to receive and engage a corresponding threading on a plate set fastener shaft 1941 (shown in FIG. 28) to securely fasten the anterior plate 220 to the posterior plate 210.

The anterior plate 220 may include one or more anterior graft chambers 233, one or more coupler apertures 292, and surfaces 231. The anterior plate 220 may include a face 221. The coupler aperture 292 may pass through the face 221 and connect to the anterior chamber 232 (as seen in FIG. 28). The opening of the coupler aperture 292 on the posterior wall of the anterior chamber 232 may have a smaller diameter than the opening of the coupler aperture 292 on the face 221, so as to allow the head of the plate set fastener 194 to pass through the face 221 and contact and engage the posterior wall of the anterior chamber 232 when fastening the anterior plate 220 to the posterior plate 210. This configuration allows for introduction of graft tissue into the anterior graft chamber through the coupler aperture 292 after the plate set fastener 194 has been installed. The surfaces 231 may be substantially the same as the surfaces 212 on the posterior plate 210.

The anchoring plate 260 may include one or more fastener apertures 261 that receive and hold a corresponding bone fastener 270. The fastener aperture 261 may include, for example, a threading that engages a corresponding threading on the bone fastener 270; or, the fastener aperture 261 may have a diameter that is greater than the diameter of the bone fastener 270, so as to allow a shaft of the bone fastener 270 to pass through the aperture unobstructed. The bone fastener 270 may be substantially the same as the bone fastener 170.

The anchoring plate 260 may be fixedly (or removably) attached to the actuator 240, or the anchoring plate 260 may be integrally formed with the actuator 240. The anchoring plate 260 may include one or more coupler pass-throughs 262 that allow the plate set fastener 194 to be substantially completely installed (shown in FIG. 28) without obstructing the pathway of the plate set fastener shaft 1941. The pass-through 262 may have any shape that does not interfere with installation of the plate set fastener 194 to fasten the anterior plate 220 to the posterior plate 210 (shown in FIG. 28).

Where the cage system 200 is configured to receive a pair of plate set fasteners 194, as shown in FIGS. 26-28, the anchoring plate 260 may include a second pass-through 262 (shown in FIGS. 27-28). The second pass-through 262 (shown in FIGS. 27-28) may be located opposite and substantially diagonally across from the pass-through 262 shown in FIG. 26, so as to allow unobstructed rotation of the anchoring plate 260 about the longitudinal axis of the actuator 240 in one direction (e.g., clockwise direction shown in FIGS. 26-28), but not the other direction when the couplers 241 are installed.

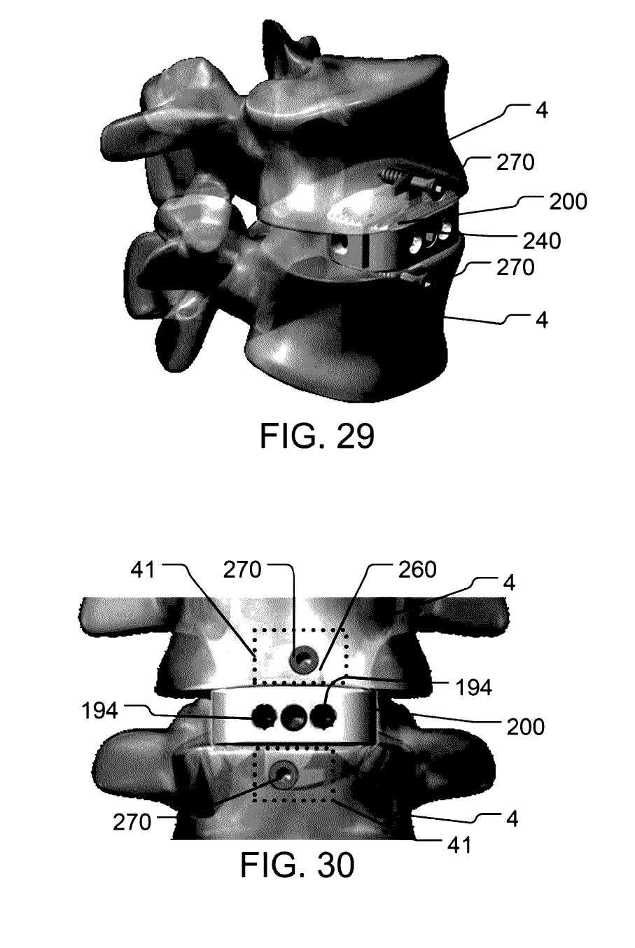

FIGS. 29-30 illustrate installing the cage 200 in a patient. Referring to FIGS. 29 and 30, after a surgical area is cleaned on a patient, an incision made, muscle tissue and/or organs moved to the side(s), and other common surgical procedures carried out, a disc may be incised, removed, and the space prepared for implanting of a cage system. The bone surfaces and edges on the adjacent vertebrae may be carefully contoured, as appropriate.

Following a discectomy procedure, a medical professional may determine an appropriate size of the cage system 200 by selecting an appropriately dimensioned cage system 200 based on, for example, height, width, depth, number of graft chambers, configuration of graft chambers, configuration of outer surface 212 (including bone interface members), and the like. Upon selecting the appropriate cage system 200, one or more of an ACIF, ALIF, or the like may be performed by placing the cage system 200 between adjacent vertebrae 4 in the space formed by the removed degenerated disc (shown in FIGS. 29-30).

Referring to FIGS. 26-28, if not already assembled, the anterior plate 220 may be fixed to the posterior plate 210 by, for example, installing plate set fasteners 194 through the openings 292 and into the coupler apertures 291 using a driver tool (not shown). The actuator 240 may be turned counterclockwise (or clockwise) using the same (or a different) driver tool to position the anchoring plate 260 in the disengaged position (shown in FIG. 27).

The cage system 100 may be placed into the space between the vertebrae 4. Then, using the driver tool (not shown) the actuator 240 may be turned clockwise (or counterclockwise) to position the anchoring plate 260 in the engaged position (shown in FIGS. 29-30).

Once the cage system 200 is properly installed with respect to the vertebrae (e.g., as seen in FIGS. 29-30), a medical professional may prepare one or more bores or holes in the vertebra 4 intended to receive bone fasteners 270. In this regard, hard bone surface may be removed and a guide track may be inserted under x-ray guidance into the vertebrae 4. An incision may be made in at least one of the adjacent vertebrae 4 to form a cutout 41 (shown in FIG. 30). The depth and position of the guide track may be checked. The depth and position of the cutout(s) 41 may be checked. The cutout 41 should be sufficiently large enough to receive and house an end portion of the anchoring plate 260 (shown in FIG. 30), but small enough to facilitate efficient and effective bone fusion.

Where the bone fastener 270 includes a bone screw, a thread may be tapped into the bone to form a tap (not shown) to receive and securely hold the bone fastener 270. The process would be repeated for each bone fastener 270. Such holes may be formed with the aid of a separate drill guide (not shown) positioned proximate or abutting vertebra 4 and inserting a drill therethrough. Alternatively, such holes may be formed free hand, without the use of a drill guide.

After the cage system 200 is properly installed with respect to the vertebrae 4 (e.g., as shown in FIG. 30), the bone fastener(s) 270 may be installed. In this regard, a driver tool (not shown), as is known by those skilled in the art, may be used to turn and drive the bone fastener(s) 270 into the vertebrae 4. It is noted that the bone fastener(s) 270 may be aligned with the tap (not shown) in the bone and screwed into the threaded tap.

Alternatively, the bone fasteners 270 may be partially installed in the tap before being contacted by the driver tool. Once the bone fasteners 270 are implanted in the desired position, the driver tool may be removed and the process repeated for each bone fastener 270.

As discussed above, the graft chamber(s) 232 and/or 233 (e.g., shown in FIGS. 26-28) may be filled with a radiolucent material such as tissue grafts. For instance, the graft chamber(s) 232 and/or 233 may be packed with bone graft. Bone graft material may facilitate bone and tissue ingrowth in and through the cage system 200. Accordingly, bone graft may promote fusion, i.e., the joining of two or more vertebrae 4. The plate set fasteners 194 may be checked to ensure they are properly tightened.

The cage system 200, including the posterior plate 210 and anterior plate 220, may be configured such that bone graft material packed within cage system 200 may be retained therein.

After the bone graft materials are installed, and the bone fasteners 270 are securely and properly placed in corresponding taps, and the installation of the cage system 200 completed, the area may be cleaned, checked, closed and other post-operative procedures carried out, as is known in the art.

As with the cage system 100 discussed above, placement of the cage system 200 within the spinal column may prevent spaces between adjacent vertebrae 4 from collapsing, thereby preventing adjacent vertebrae from resting immediately on top of one another and inducing fracture of vertebra 4, impingement of the spinal cord, and/or pain. Additionally, the cage system 200 may facilitate fusion between adjacent vertebrae 4 by stabilizing adjacent vertebrae 4 relative to one another.

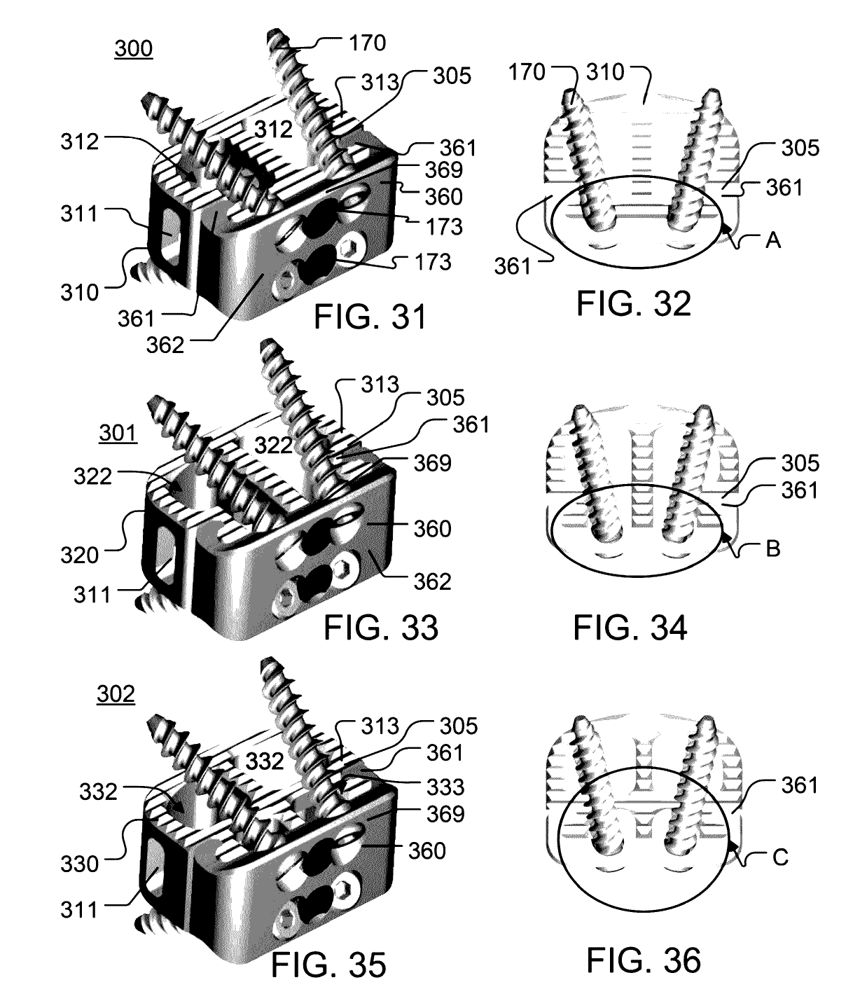

FIGS. 31, 33, and 35 illustrate perspective views of cage systems 300, 301, and 302, respectively, including a plating device. As seen in FIGS. 31, 33, and 35, the plating device may include an intradiscal plate 360. The cage systems 300, 301, and 302 are shown with bone fasteners 170.

FIGS. 32, 34, and 36 illustrate top (or bottom) views of the cage systems 300, 301, and 302, respectively, including the intradiscal plate 360 and bone fasteners 170. The cage systems 300, 301, 302 include cage bodies 310, 320, 330, respectively. The cage bodies 310, 320, 330 may include surfaces 313, which may be substantially the same as surface 112 (shown in FIG. 3A). Each of the cage bodies 310, 320, 330 may include one or more plate interfaces 305. The plate interface 305 is configured to receive a corresponding cage connector 361 (shown in FIGS. 32, 34, 36). The plate interface 305 may be constructed as a recess or grove in the cage body 310 (320, 330) that corresponds to and matches a male portion of the cage connector 361, as shown. Accordingly, the intradiscal plate 360 may be interchangeably used with the cage bodies 310, 320, or 330.

As seen in FIGS. 31-36, the cage bodies 310, 320, 330 may include one or more graft chambers 312, 322, 332, respectively. The walls that form the graft chambers 312, 322, 332, may include one or more apertures (or windows) 311. The cage body 310, for example, may be designed as a closed configuration having a pair of graft chambers 312 formed by the inner walls of the cage body 310, including the inner wall shown in the circled area A. The cage body 320 may be designed as an open configuration having a pair of graft chambers 322 formed by the inner walls of the cage body 320 and the inner wall of the intradiscal plate 360, as seen in the circled area B in FIG. 34. The cage body 330 may be designed as a hybrid configuration having a pair of posterior graft chambers 332 formed by the inner walls of the cage body 330, and a pair of anterior graft chambers 333 formed by inner walls of the cage body 330 and the inner wall of the intradiscal plate 360, as seen in the circled area C in FIG. 36.

The intradiscal plate 360 has a face 362 that includes one or more apertures (e.g., two, three, four, or more) for corresponding bone fasteners 170. The plate 360 may include one or more bone interfaces 369, such as, for example, one bone interface 369 along an upper edge of the intradiscal plate 360, and/or one bone interface 369 along an lower edge of the plate 360. The bone interface 369 is configured to contact and seat against an edge and/or a surface portion of an adjacent vertebra 4, so as to provide proper and secure positioning of the cage system 300 (301, 302) with respect to the vertebrae 4.

The cage systems 300, 301, 302 may include one or more bone fastener locks 173, so as to secure the bone fastener(s) 170 against unscrewing or withdrawing from the cage system 300 (301, 302), as discussed above. The cage systems 300, 301, 302, may further include blocking element 174 and/or blocking element cutout 175, as discussed above.

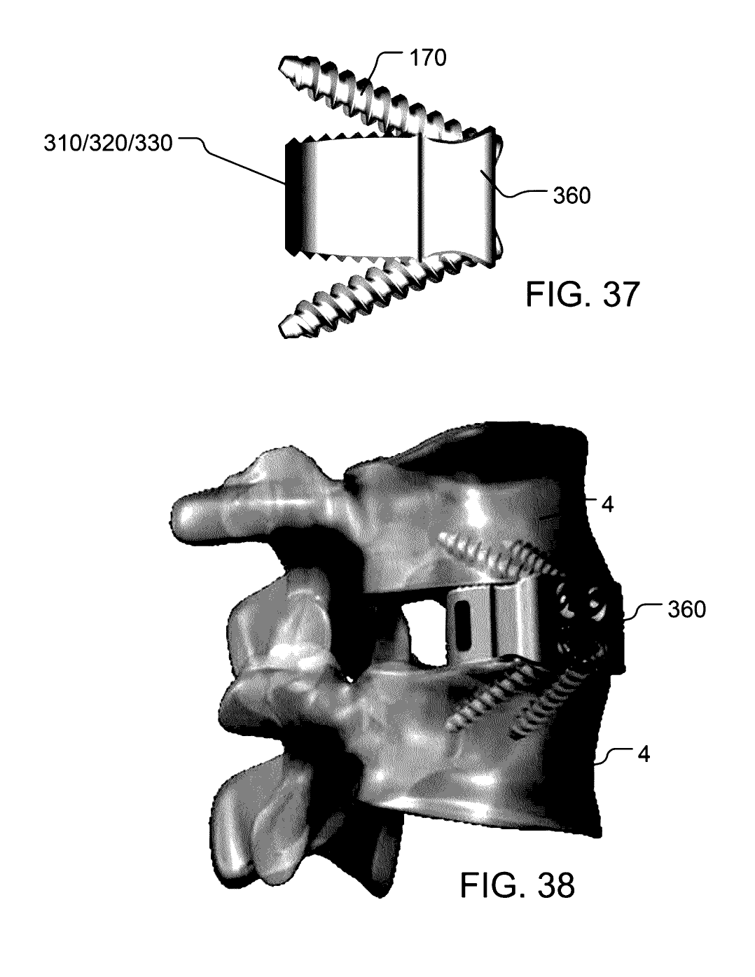

FIG. 37 illustrates a side view of the cage systems 300, 301, 302, shown in FIGS. 31-36.

FIG. 38 illustrates the cage system 300 (301, 302) installed in a patient. As discussed above, following a discectomy procedure, a medical professional may determine an appropriate size of the cage system 300 (301, 302) by selecting an appropriately dimensioned cage body 310 (320, 330) (e.g., in terms of height, width, depth, shape, etc.). Upon determining the appropriate cage body 310 (320, 330), an intradiscal plate 360 may be selected that matches the size of the cage body 310 (320, 330). One or more holes or apertures may be drilled into one or more of the vertebrae 4 to receive corresponding bone fasteners 170.

Once the cage body 310 (320, 330) and plate 360 are selected, one or more of an ACIF, ALIF, or the like may be performed by placing the cage body 310 (320, 330) between adjacent vertebrae 4 in the space formed by the removed degenerated disc. The plate 360 may be adjusted so as to contact and properly seat against the edges of the adjacent vertebrae 4 (shown in FIG. 38). After proper positioning, the bone fasteners 170 may be inserted into the vertebra 4 through the apertures provided in the plate 360, thereby securely fastening the cage 300 (301, 302) to the vertebra(e) 4.

As with the implants discussed above, placement of the cage system 300 (301, 302) within the spinal column may prevent spaces between adjacent vertebrae 4 from collapsing, thereby preventing adjacent vertebrae from resting immediately on top of one another and inducing fracture of vertebra 4, impingement of the spinal cord, and/or pain. Additionally, the cage system 300 (301, 302) may facilitate fusion between adjacent vertebrae 4 by stabilizing adjacent vertebrae 4 relative to one another.

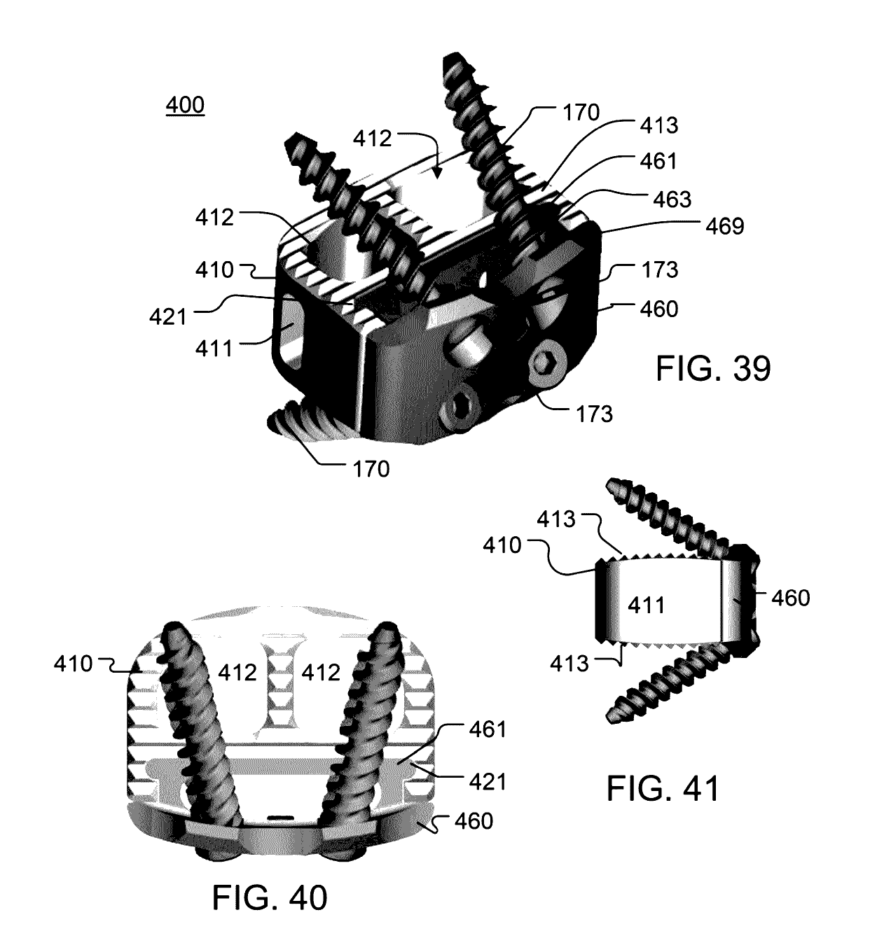

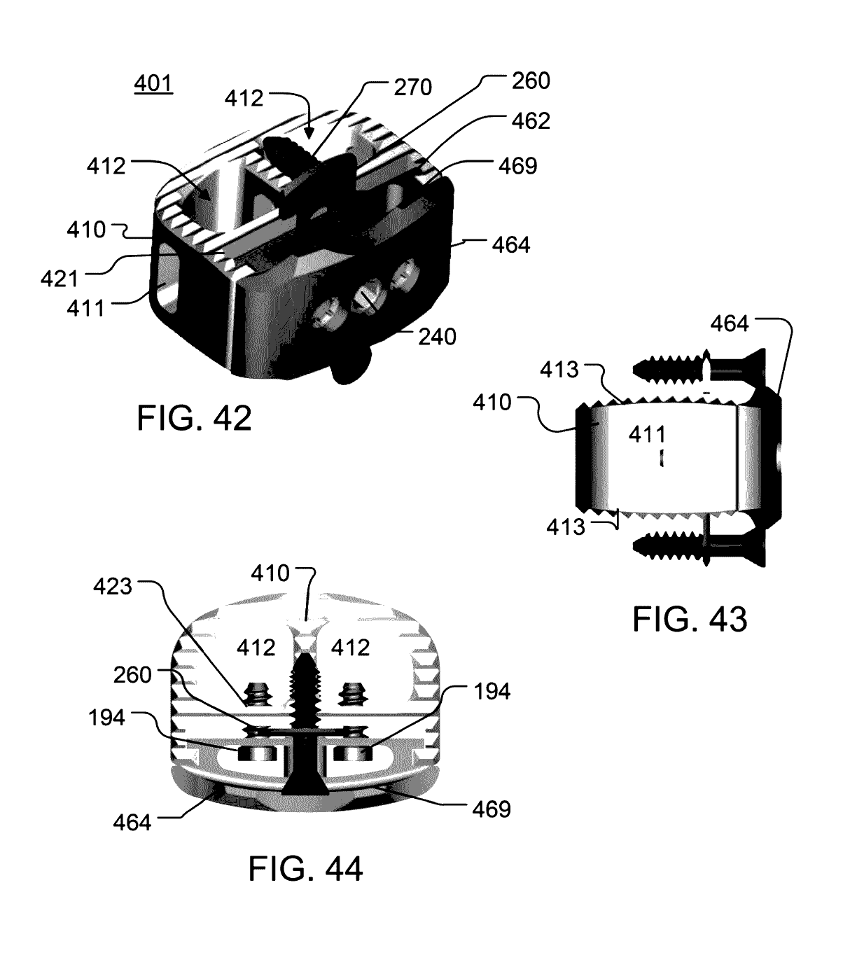

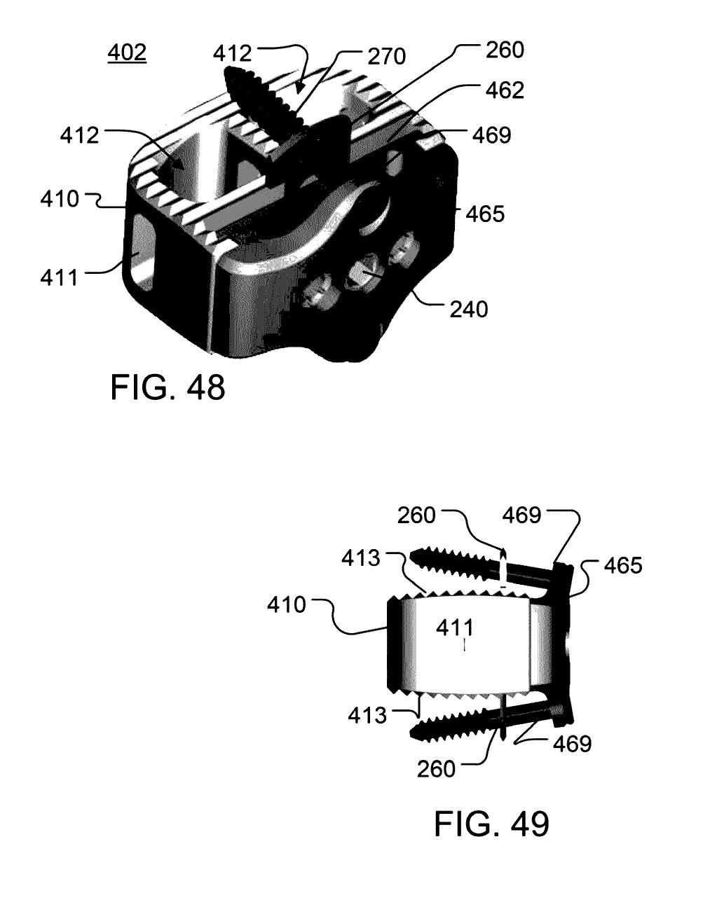

FIGS. 39, 42, and 48 illustrate perspective views of cage systems 400, 401, and 402, respectively; FIGS. 40 and 44 illustrate top views of the cage systems 400 and 401, respectively; and, FIGS. 41, 43, and 49 illustrate side views of the cage systems 400, 401, and 402, respectively. As seen in FIGS. 39-44 and 48-49, the cage systems 400, 401, and 402 may include the same cage body 410, but differing intervertebral plates 460 (shown in FIGS. 39-41), 464 (shown in FIGS. 42-44), and 465 (shown in FIGS. 48-49).

Referring to FIGS. 39-44 and 48-49, the cage body 410 includes one or more graft chambers 412, upper and lower surfaces 413, and a plate interface 421. The graft chambers 412 may be formed by inner walls of the cage body 410. The upper and lower surfaces 413 may be substantially the same as upper and lower surfaces 112 (shown in FIG. 3A). The plate interface 421 may be configured to receive and hold different sizes and shapes of cage interfaces 461 (shown in FIG. 41) or 462 (shown in FIGS. 42, 48).

The cage body 410 may include one or more apertures (or windows) 411 in the walls of the cage body 410 that form the one or more graft chambers 412. The apertures 411 may allow, for example, blood, tissue, and bone to flow into the graft chamber(s) 412 from the surrounding area around the cage body 410.

The cage body 410 may include one or more coupler apertures 423 that receive corresponding one or more plate set fasteners 194, as seen in FIG. 44. The coupler aperture(s) 423 may be substantially the same as the coupler aperture 291 (shown in FIG. 28). The coupler aperture(s) 423 may include threading that engages threading on a corresponding plate set fastener 194. Alternatively, the coupler aperture(s) 423 may include an opening having an inner diameter greater than the outer diameter of the plate set fastener shaft 1941, so as to allow the plate set fastener shaft 1941 to pass therethrough unobstructed.

The intradiscal plate 460 (shown in FIGS. 39-41) has a face that may include one or more apertures configured to receive corresponding bone fasteners 170. The cage system 400, including intradiscal plate 460, may include one or more bone fastener locks 173 to secure and prevent the bone fastener(s) 170 from withdrawing or unscrewing. The cage system 400 may further include blocking element 174 (not shown) and/or blocking element cutout 175 (not shown), as discussed above.

The intradiscal plate 460 may include one or more anterior graft chambers 468 (shown in FIG. 40) that are designed to hold bone graft. The anterior graft chambers 468 may be configured to allow portions of the bone fastener(s) 170 to pass therethrough for a more compact, lower profile design of the intradiscal plate 460, as seen in FIGS. 39-41.

The intradiscal plate 464 (shown in FIGS. 42-44) has a face that may include one or more coupler apertures 292 and an actuator aperture 293. The intradiscal plate 464 may include one or more graft chambers 468 formed by the inner walls of the intradiscal plate 464. The posterior wall of the graft chamber(s) 468 may include a coupler aperture 463 that may be aligned with a corresponding coupler aperture 423 in the cage body 410 for installation of a plate set fastener 194. The intradiscal plate 464 may include an anchoring plate 260. The anchoring plate 260 may be fixedly attached to or integrally formed with the actuator 240, so that when the actuator 240 is manipulated (e.g., turned clockwise or counterclockwise), the anchoring plate 260 rotates about the longitudinal axis of the actuator 240 from a disengaged (or engaged) position to an engaged (or disengaged) position (shown in FIGS. 42-43).

The intradiscal plate 464 may include one or more bone interfaces 469, which may be provided, for example, along the upper and/or lower edges of the intradiscal plate 464. The bone interface(s) 469 may be configured to contact an edge portion of a vertebra 4 and/or facilitate in proper positioning of the cage 401 in the implant site.

The extradiscal plate 465 may have a structure similar to that of the intradiscal plate 464, except that it is constructed for extradiscal applications and may include a bone interface 4691. The bone interface 4691 may include an aperture that receives a bone fastener 270. The bone interface 4691 may be angled as seen in FIG. 49 to maximize surface contact and seating with an edge portion of an adjacent vertebra 4.

Referring to FIGS. 39-41, when assembling the cage 400, the intradiscal plate 460 may be positioned and aligned such that the lower (or upper) surface of the cage interface 461 is above (or below) the upper (or lower) cage body surface 413 and aligned so that the cage interface 461 may be slid downward (or upward) into the intradiscal plate interface 421 until the upper (or lower) surface of the cage interface 461 is substantially flush with the upper (or lower) surface 413 of the cage body.

Referring to FIGS. 42-44, when assembling the cage system 401, the intradiscal plate 464 may be assembled in a manner similar to that described above for cage system 400, except that the process may include installing one or more plate set fasteners 194 through the face of the intradiscal plate 464, through an aperture 463 in the posterior wall of the intervertebral plate 464 and into the aperture 423 of the cage body 410. The plate set fastener(s) 194 may be installed, for example, by turning the plate set fastener(s) 194 in a clockwise (or counterclockwise) direction in the case where the plate set fastener(2) 194 is a bolt, a screw, or the like.

The cage system 402 (shown in FIGS. 48-49) may be assembled in a manner similar to that for the cage system 401.

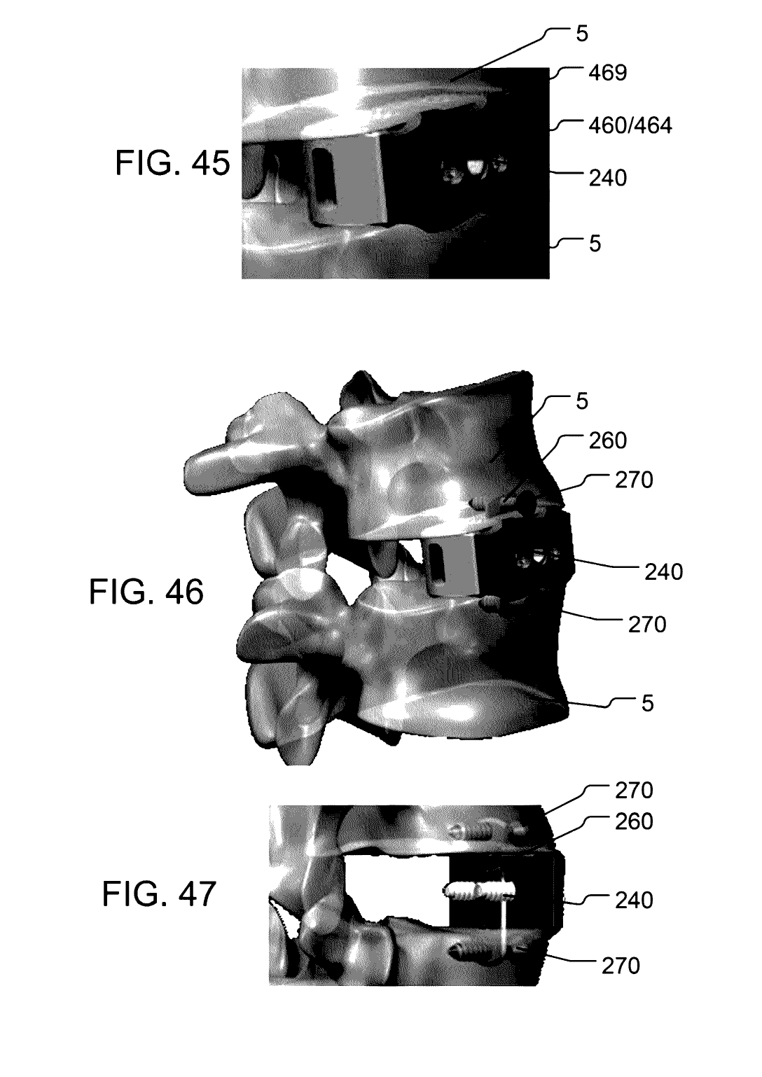

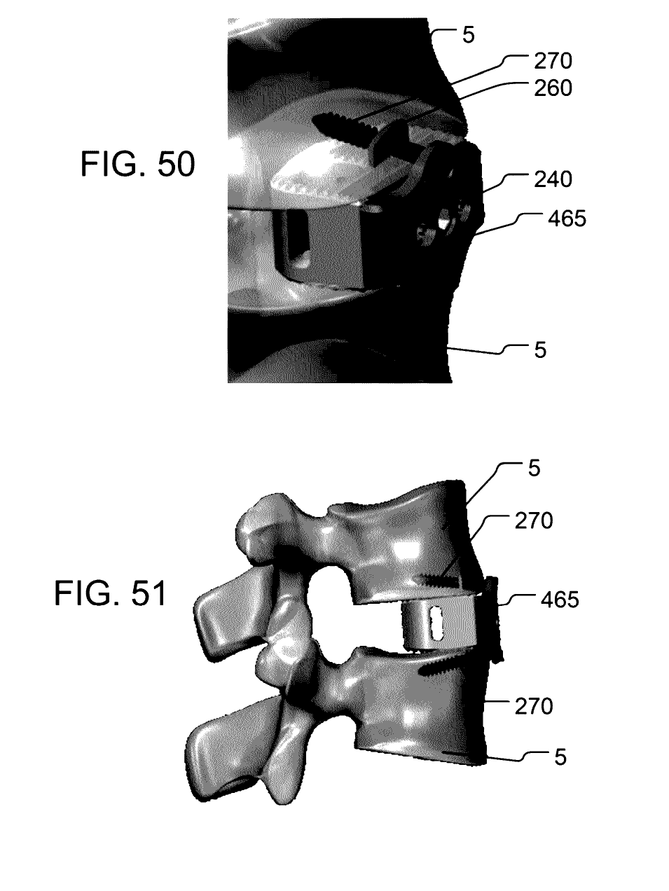

FIGS. 45-47 illustrate various stages of installing the cage system 401 (shown in FIGS. 42-44) in a patient; and, FIGS. 50-51 illustrate various stages of installing the cage system 402 (shown in FIGS. 48-49), including the extradiscal plate 465 in a patient. A similar process may be used to install the cage system 400 (shown in FIGS. 39-41) in a patient.

Referring to FIGS. 45-47, following a discectomy procedure, a medical professional may determine an appropriate size of the cage system 401 by selecting an appropriately dimensioned cage body 410 and an appropriately dimensioned intradiscal plate 464 (e.g., in terms of height, width, depth, shape, number of bone fastener apertures, shape and size of bone fastener apertures, positioning of bone fastener apertures, etc.). Upon determining the appropriate cage body 410 and intradiscal plate 464, an incision may be made in at least one of the adjacent vertebrae 4 to form a cutout 41 (shown in FIGS. 46-47). One or more holes or apertures may be drilled into one or more of the vertebrae 4 to receive corresponding bone fasteners 270. The cutout 41 should be sufficiently large enough to receive and house an end portion of the anchoring plate 260 (shown in FIGS. 46-47), but small enough to facilitate efficient and effective bone fusion.

Once the cage body 410 and intrasdiscal plate 464 are selected and the cutout(s) 41 made, one or more of an ACIF, ALIF, or the like may be performed by placing the cage 401 between adjacent vertebrae 4 in the space formed by the removed degenerated disc. After proper placement, the actuator 240 may be turned to rotate the ends of the anchoring plate 260 into corresponding cutouts 41. When properly positioned, bone fasteners 270 may be inserted into the vertebra 4, through the apertures 261, thereby securely fastening the cage 401 to the vertebra(e) 4.

Substantially the same process as the above may be carried out for implanting of the cage system 402 in FIGS. 50-51, except that the bone fastener(s) 270 are first inserted through an aperture in the extradiscal plate 465 and then installed through a hole in the vertebra 4 and through the aperture 261 in the anchor plate 260. It is noted that the hole(s) for the bone fastener(s) 270 may be drilled after the cage system 402 (or 401) has been implanted, properly positioned, and the anchor plate 260 manipulated to a fully engaged position (shown in FIG. 50).

The terms “including,” “comprising,” and variations thereof, as used in this disclosure, mean “including, but not limited to,” unless expressly specified otherwise.

The terms “a,” “an,” and “the,” as used in this disclosure, means “one or more,” unless expressly specified otherwise.

Devices that are in communication with each other need not be in continuous communication with each other, unless expressly specified otherwise. In addition, devices that are in communication with each other may communicate directly or indirectly through one or more intermediaries.

Although process steps, method steps, algorithms, or the like, may be described in a sequential order, such processes, methods and algorithms may be configured to work in alternate orders. In other words, any sequence or order of steps that may be described does not necessarily indicate a requirement that the steps be performed in that order. The steps of the processes, methods or algorithms described herein may be performed in any order practical. Further, some steps may be performed simultaneously.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device may be alternatively embodied by one or more other devices which are not explicitly described as having such functionality or features.

While the disclosure has been described in terms of exemplary embodiments, those skilled in the art will recognize that the disclosure can be practiced with modifications in the spirit and scope of the appended claims. These examples are merely illustrative and are not meant to be an exhaustive list of all possible designs, embodiments, applications or modifications of the disclosure.

What is claimed is:

1. A cage for implanting in bone, comprising:
a first plate having a surface that contacts a first bone surface;
a second plate having a surface that contacts a second bone surface;
an intermediary plate that dynamically couples to the first plate and the second plate;
an actuator that drives and causes the intermediary plate to move between the first plate and the second plate along a predetermined direction; and
an anchor that attaches to the first plate and the second plate to engage the actuator to drive the actuator longitudinally along the predetermined direction
wherein at least one of the first plate, second plate, and intermediary plate has a plate connector interface that receives and rotably engages and alignably locks to a corresponding intermediary plate connector of a plating device.

2. The cage of claim 1, wherein at least one of the first plate and second plate comprises a guide track that engages and guides the intermediary plate as it moves between the first plate and the second plate along the predetermined direction.

3. The cage of claim 2, wherein the intermediary plate comprises a guide that engages the guide track to go guide the intermediary plate as it moves between and along inner surfaces of the first plate and the second plate in the predetermined direction.

4. The cage of claim 1, wherein the anchor comprises an anchor lock that engages the first plate or the second plate to prevent the anchor from moving.

5. The cage of claim 4, wherein the moving comprises rotation of the anchor about a longitudinal axis of the actuator.

6. The cage of claim 1, wherein inner walls of the first plate, second plate and intermediary plate form a graft chamber.

7. The cage of claim 4, wherein at least one of the first plate and the second plate comprises a receiver that holds the anchor lock.

8. The cage of claim 1, further comprising:
a pin that engages an anterior portion of the intermediary plate.

9. The cage of claim 8, wherein the pin engages a portion of the actuator to substantially affix the actuator to the intermediary plate.

10. A cage for implanting in bone, comprising:
a first plate having a surface that contacts a first bone surface;
a second plate having a surface that contacts a second bone surface;
an intermediary plate that movably attaches to the first plate and the second plate; and
an actuator that drives and causes the intermediary plate to move between the first plate and the second plate along a predetermined direction.

11. The cage of claim 10, further comprising:
an anchor that engages the actuator to drive the actuator longitudinally along the predetermined direction, or in a direction substantially opposite to the predetermined direction.

12. The cage of claim 10, wherein at least one of the first plate and second plate comprises a guide track that engages and guides the intermediary plate as it moves along the predetermined direction between the first plate and the second plate.

13. The cage of claim 12, wherein the intermediary plate comprises a guide that engages the guide track to go guide the intermediary plate as it moves between the first plate and the second plate along the predetermined direction.

14. The cage of claim 11, wherein the anchor comprises an anchor lock that engages at least one of the first plate and the second plate to prevent the anchor from moving.

15. The cage of claim 11, wherein the moving comprises rotation of the anchor about a longitudinal axis of the actuator.

16. The cage of claim 10, wherein inner walls of the first plate, second plate and intermediary plate form a graft chamber.

17. The cage of claim 14, wherein at least one of the first plate and the second plate comprises a receiver that holds the anchor lock.

18. The cage of claim 10, further comprising:
a pin that engages and holds an anterior portion of the intermediary plate with respect to a portion of the actuator.

19. A cage for implanting in bone, comprising:
a first plate having a surface that contacts a bone surface;
an intermediary plate that movably attaches to the first plate; and
an actuator that drives and causes the intermediary plate to move with respect to the first plate along a predetermined direction.

20. The cage of claim 19, further comprising:
a second plate having a surface that contacts another bone surface,
wherein the intermediary plate movably attaches to the second plate.
